(19) United States
(12) Patent Application Publication
(10) Pub. No.: US 2004/0087985 A1
(43) Pub. Date: May 6, 2004

(54) Graft and connector delivery

(76) Inventors: Amir Loshakove, Moshav-Bazra (IL); Ido Kilemnik, Herzlia (IL); Tachum Feld, D N Izrael (IL); Dvir Keren, Petach-Tikva (IL); Eitan Konstantino, Pleasanton, CA (US)

Correspondence Address: William H Dippert, Reed Smith, 599 Lexington Avenue 29th Floor, New York, NY, 10022-7650, US

(21) Appl. No.: 10/239,387

(22) PCT Filed: Mar. 20, 2001
(86) PCT No.: PCT/IL01/00266

(51) Int. Cl. A61B 17/08

(52) U.S. Cl. 606/153

(57) ABSTRACT

A vascular attachment device (800) seals an opening between two blood conduit tips, where the device comprises a ring element (752); a plurality of fingers (754) mounted on the ring element (752) and adapted to seal at least a portion of an opening between the two blood conduit lips by compressing at least two lips between at least one finger (754) and the ring element (752); and at least one puller spike (812) adapted for pulling, inside the body, at least one of the lips to a space defined between the finger (754) and the ring element (752).

### RELATED APPLICATIONS

The present application is related to the following PCT applications filed by applicants Bypass Inc., et al., PCT/IL99/00285, PCT/IL99/00284, PCT/IL99/00674, PCT/IL99/00670, PCT/IB00/00302 and PCT/IB00/00310, and PCT/IL00/00609, PCT/IL00/00611, PCT/IL01/00074 and an application filed on even date as the instant application, in the Israel Receiving Office of the PCT, titled “TRANSVASCULAR BYPASS METHOD AND SYSTEM” and having attorney docket 088/02021, all of which designate the US, the disclosures of which are incorporated herein by reference.

### FIELD OF THE INVENTION

The present invention relates to the manipulation of vessel hole lips, especially for effecting anastomosis connections.

### BACKGROUND OF THE INVENTION

Eversion of vessel lips is typically performed outside the body, for example as described in U.S. Pat. Nos. 5,366,462 and 5,695,504, the disclosure of which is incorporated herein by reference. However, the lips of a hole in an aorta cannot be thus manipulated, since the aorta must remain in the body.

### SUMMARY OF THE INVENTION

An aspect of some embodiments of the invention relates to eversion of the lips of a hole in a blood vessel, for example so that they are engaged by an anastomotic connector or a hole closure device. In an exemplary embodiment of the invention, a puller is used to retract the lips into the connector. Optionally, the puller is removable, for example being part of a device delivery system. Alternatively, the puller may remain in the connection. Alternatively, no puller is used, for example, a change in the device geometry causing the lips to be retracted. Alternatively to retraction into the device, the retraction is into the delivery system, after which the anastomotic device is applied. The delivery system, in some embodiments of the invention, guides the retraction.

In an exemplary embodiment of the invention, the pullers transfix the lip. Alternatively, the pullers only engage the lip.

The pullers may be used in various types of vascular devices, whereby the pullers bring the lips of the blood vessel into a desired location relative to another lip or a device, and optionally hold the lip in place, or move it, during an operation of the device. Optionally, more than one set of pullers is provided, for example, for manipulating multiple vessels. Where a plurality of pullers are provided, the pullers may, for example, act simultaneously or in sequence.

The anastomotic device may have, for example, one part, two parts or may comprise a plurality of independent (or attached by a thread) clamping elements.

The retracted lips may be pressed against each other to prevent blood leakage. Alternatively, they may be pressed against a part of the device.

An aspect of some embodiments of the invention relates to the design of an anastomotic connector. In an exemplary embodiment of the invention, the connector comprises a ring and a plurality of fingers attached to the ring and adapted to engage one or more blood vessel lips, by friction between the fingers and the lips. Optionally, two sets of fingers are provided for engaging the lips, one on each side of the ring. Alternatively, only one set of fingers is provided. Optionally, the fingers do not pierce the lips, for example, having blunt tips or contacting the lips with their side. The fingers may be, for example, plastically deformable or they may be elastic, shape memory and/or super-elastic. Optionally, the fingers hold two lips together, for example, lips of a same blood vessel or lips of two different blood vessels. Alternatively or additionally to holding the lips against the same or other fingers, the fingers hold the lips against the ring. Optionally, the fingers fold inwards, towards the ring.

An aspect of some embodiments of the invention relates to a method of performing an anastomosis connection. In an exemplary embodiment of the invention, a connector delivery device has three states. A first state in which a plurality of spikes are arranged to be inserted into an opening in a blood vessel. In a second state, the spikes are retracted, pulling the blood vessel towards a graft loaded in the delivery device. In a third state, the spikes are pulled back so that they tear off. In an exemplary embodiment of the invention, the spikes are attached to an end of a tube that is retracted by the delivery system. In an optional third state, further retraction of the spikes splits apart the delivery system, for example, by a knife or a protrusion on the retracted spike tube.

In an exemplary embodiment of the invention, the spikes are ripped off by being pulled against a first apertured ring, the ring having apertures that are smaller than protrusions on the spikes. Optionally, the apertures are slots in the ring and the spikes are restrained from moving sideways in the slots by a second apertured ring. Optionally, the second apertured ring serves as a base ring for holding together the ripped spikes. Optionally, the base ring is thinner than the first apertured ring.

A similar device may be used for sealing a hole, for example, if the ring compresses radially, twists and/or if the ring is a sealed circle.

An aspect of some embodiments of the invention relates to a method of attaching two blood vessel using multiple clips. In an exemplary embodiment of the invention, the lips of the two blood vessels are everted and the clips are closed onto the everted part, so that the clips remain outside the blood vessels and there is an intima-to-intima connection between the blood vessels. In some embodiments of the invention, the lips are everted using hooks that transfix the lips. Alternatively, the hooks do not transfix the lips.

An aspect of some embodiments of the invention relates to a method of mounting a graft on a connector, such that graft parts that are folded back lie between and adjacent to forward spikes, rather than being transfixed by the spikes. The parts of the graft may be held in place by removable spikes while an anastomosis to a target vessel is being performed. Alternatively, the connector may include a second set of spikes, which hold the graft parts in place.

An aspect of some embodiments of the invention relates to a method of inserting spikes of an anastomosis connector into a target vessel. In general, when the forward spikes of the connector are thin, long and hooked at their ends, they are susceptible to interlocking, which interlocking prevents proper deployment of the connector. Such interlocking is more likely to occur if the insertion of the spikes into the target vessel is rushed, for example, if blood is spurting out of the target vessel or the target vessel is sealed.

In an exemplary embodiment of the invention, a two step process is provided. In a first step, a cut is made in the vessels and a guide is inserted into the thus formed cut. In a second step, the forward spikes are inserted through the guide, the guide is removed and the connection is performed by retracting the spikes. Optionally, the guide is removed by it being a tearable tube that is retracted over a delivery system, which retraction tears the guide.

There is thus provided in accordance with an exemplary embodiment of the invention, a vascular attachment device for sealing an opening between two blood conduit lips, comprising:

a ring element;

a plurality of fingers mounted on said ring element and adapted to seal at least a portion of an opening between two blood conduit lips by compressing said at least two lips between a finger and at at least one of said finger and said ring; and

at least one puller spike adapted for pulling, inside the body, at least one of said lips to a space defined between said finger and said ring. Optionally, said fingers are restrained back from a resting position in which they engage said lip in said space. Alternatively or additionally, said at least one puller is integral with an elongate retractable tube. Alternatively or additionally, said at least one puller comprises a plurality of pullers arranged in the form of a cone, an apex of said cone being adapted for inserting into an opening in a blood vessel. Optionally, said at least one puller is outside of said ring such that when said pullers are retracted, the cone opens up. Alternatively or additionally, each puller comprises a bent tip, adapted to engage said lip.

In an exemplary embodiment of the invention, each puller comprises a designated tear area, for tearing said puller after it is retracted towards said ring, so that only a tip portion of said puller remains in the body. Optionally, said tip portion is pre-curled such that said tearing allows said portion to revert to a curled closed state. Alternatively or additionally, each puller comprises a trans-axial protrusion for stopping retraction of said tip portion.

In an exemplary embodiment of the invention, each puller is smooth, to allow retraction of said puller through said lips and out of said body.

Alternatively or additionally, each puller has a sharp tip adapted for insertion through a graft wall.

In an exemplary embodiment of the invention, said ring has the shape of an ellipse.

In an exemplary embodiment of the invention, said fingers do not penetrate any of said lips.

There is thus provided in accordance with an exemplary embodiment of the invention, a vascular attachment device for sealing an opening between two blood conduit lips, comprising a plurality of bendable clips, said clips being adapted for gripping two lips between them and for sealing said opening by forcing said lips towards each other, wherein said clip elements are blunt and do not penetrate said blood conduit walls. Optionally, said clips are arranged on a ring.

There is also provided in accordance with an exemplary embodiment of the invention, a vascular attachment device for sealing an opening between two blood conduit lips, comprising:

a ring element defining a plurality of apertures;

a plurality of puller spikes having tips and defining designated tear areas near said tips, said tips being adapted to fit through said apertures and integral with a retractable elongate tube, such that when said device is deployed only said tips of said spikes remain in said body. Optionally, said tube is an axially split tube. Alternatively or additionally, said tube comprises a protrusion adapted for axially splitting a matching delivery system, when said tube is sufficiently retracted. Alternatively or additionally, said spikes are pre-curled, such that when said spiked are torn at said designated tear areas, said tips revert to a pre-curled state having a greater curl arc angle than prior to said tearing. Alternatively or additionally, said apertures define leaf elements for preventing reverse motion of said spikes. Alternatively or additionally, said spikes define a protrusion on said spikes adjacent said designated tear areas.

There is also provided in accordance with an exemplary embodiment of the invention, a connector delivery system, comprising:

a retractor;

a tube integral with a plurality of puller spikes of said connector, said tube coupled to said retractor for retraction thereby, said spikes defining at least one thickened areas on at least one spike; and

a base ring for preventing said at least one thickened areas from retracting, thereby causing said spikes to tear when said retractor retracts said tube a sufficient amount. Optionally, said tube comprises a protrusion and wherein said delivery system is adapted to be split by said protrusion when said tube is sufficiently retracted. Alternatively or additionally, said system comprises a stationary tube for maintaining said base ring in place relative to said integral tube.

There is also provided in accordance with an exemplary embodiment of the invention, a connector delivery system for delivering a ring connector having a plurality of fingers, said fingers defining an open configuration and a closed configuration mounted thereon, comprising:

a retractor

a tube integral with a plurality of puller spikes and coupled to said retractor for retraction thereby; and

an outer tube adapted to close a plurality of said fingers, when said puller spikes are retracted into said ring connector. Optionally, said outer tube defines a plurality of slots, for guiding a straightening of said puller spikes, when said puller spikes are retracted past said connector.

In an exemplary embodiment of the invention, said fingers close plastically. Optionally, said outer tube has an inner lip with an inner diameter smaller than an outer diameter of said connector, such that when said outer tube is moved relative to said connector, said fingers are pushed inwards by the inner lip towards said ring.

In an exemplary embodiment of the invention, said fingers close to said closed configuration by said fingers being released. Optionally, said outer tube defines an inner lip, against which said fingers are held away from said ring, such that when said outer tube is retracted, said fingers are released from said lip and close. Alternatively, said outer tube defines a plurality of slots, said fingers being held in said slots, such that when said outer tube is retracted, said fingers are released from said slots and close. Optionally, said slots have a width said slot width being narrower than a width of said fingers. Optionally, said slot width is less than 10% narrower than said finger width.

In an exemplary embodiment of the invention, said system comprises a stationary tube for maintaining said connector in place relative to said integral tube.

There is also provided in accordance with an exemplary embodiment of the invention, a method of pulling back fingers of a ring connector, in preparation for performing an anastomosis connection, comprising:

providing a connector delivery system including a slotted outer tube, said connector being mounted inside said tube, such that said fingers match up with said slots;

inserting a tool into a slot to be guided by said slot and to contact said finger; and

pulling back said fingers using said tool, to be held by said slotted outer tube. Optionally, said fingers are pulled back to lie in said slots. Alternatively, said fingers are pulled back using a tool inserted through said slots, such that said fingers lie within an inner lip of said outer tube.

There is also provided in accordance with an exemplary embodiment of the invention, apparatus for anastomosis, comprising:

a delivery system includes conical shaped arrangement of puller spikes;

a cone shaped body defining an opening at either end, a wide opening, at abase thereof, for receiving said conical arrangement and a narrow opening, at an apex thereof, for insertion into a blood vessel. Optionally, said cone shaped body is so shaped that when said delivery system is advanced, said narrow opening widens. Alternatively or additionally, said cone shaped body is pre-split axially. Alternatively or additionally, said apparatus comprises a cutting mechanism adapted to fit in said cone and comprising at least one cutting blade that fits through said narrow opening said cone. Optionally, said apparatus comprises a smaller, base-first cone having an apex meeting said apex of said cone, for inserting in a hole cut by said at least one blade, such that said smaller cone and said cone define a saddle.

In an exemplary embodiment of the invention, said cone is at least partially pre-split from an apex thereof. Optionally, said cone is pre-split on opposite sides.

### BRIEF DESCRIPTION OF THE FIGURES

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements or parts which appear in more than one figure are preferably labeled with a same or similar number in all the figures in which they appear, in which:

FIGS. 1A-1D illustrate a blood vessel attachment method and apparatus, in accordance with an exemplary embodiment of the invention;

FIG. 1E is a top view of a clip suitable for the method illustrated in FIGS. 1A-1D;

FIGS. 2A-2B illustrate a blood vessel attachment method, in accordance with an alternative exemplary embodiment of the invention;

FIG. 2C illustrates an alternative blood vessel attachment device, in accordance with an alternative exemplary embodiment of the invention;

FIG. 2D illustrates an alternative clip, in accordance with an exemplary embodiment of the invention;

FIGS. 3A-3D illustrate a hole-closure device based on a clip-puller combination, in accordance with an exemplary embodiment of the invention;

FIG. 4 illustrates a multi-clip connector, in accordance with an exemplary embodiment of the invention;

FIGS. 5A-5F illustrate a method of deploying the clip of FIG. 4, in accordance with an exemplary embodiment of the invention;

FIGS. 6A-6C illustrate an alternative method of deploying multiple clips in an anastomotic connection, in accordance with an exemplary embodiment of the invention;

FIG. 7 is a ring-clip anastomosis connector, in accordance with an alternative exemplary embodiment of the invention;

FIG. 8 is a cut-through view of a tip of a loaded delivery system for delivering the connector of FIG. 7, in accordance with an exemplary embodiment of the invention;

FIG. 9A is a perspective view of the tip of the loaded delivery system of FIG. 8;

FIG. 9B is a perspective view of the tip of an alternative loaded delivery system, in accordance with an exemplary embodiment of the invention;

FIG. 10A is a perspective view of the complete loaded delivery system of FIG. 8;

FIG. 10B is cut, through a side view of the complete loaded delivery system of FIG. 8;

FIGS. 11A-11E illustrate a connector in which a partial eversion is achieved, in accordance with an exemplary embodiment of the invention;

FIGS. 12A illustrates a part of an anastomotic connector, in accordance with an exemplary embodiment of the invention;

FIGS. 12B-12D illustrate a process of deploying a connector, in which part of the connector is removed;

FIGS. 12E-12G illustrate the effect of the process of FIGS. 12B-12D, for a single spike of the connector;

FIGS. 12H-12J illustrate a connector with self-curling spikes, in accordance with an exemplary embodiment of the invention;

FIGS. 13A and 13B show a connector delivery system, in accordance with an exemplary embodiment of the invention;

FIGS. 14A-14D illustrate a pair of interacting rings and their use in the system of FIG. 13;

FIG. 15 illustrates a vessel punching and penetration device, in accordance with an exemplary embodiment of the invention;

FIGS. 16A-16F illustrate a process of performing an anastomosis using the punch device of FIG. 15, in accordance with an exemplary embodiment of the invention; and

FIGS. 17A-17C illustrate the deployment of other clip-devices for the attachment of two blood vessels, in accordance with exemplary embodiments of the invention.

### DETAILED DESCRIPTION OF EXEMPLARY EMBODIMENTS

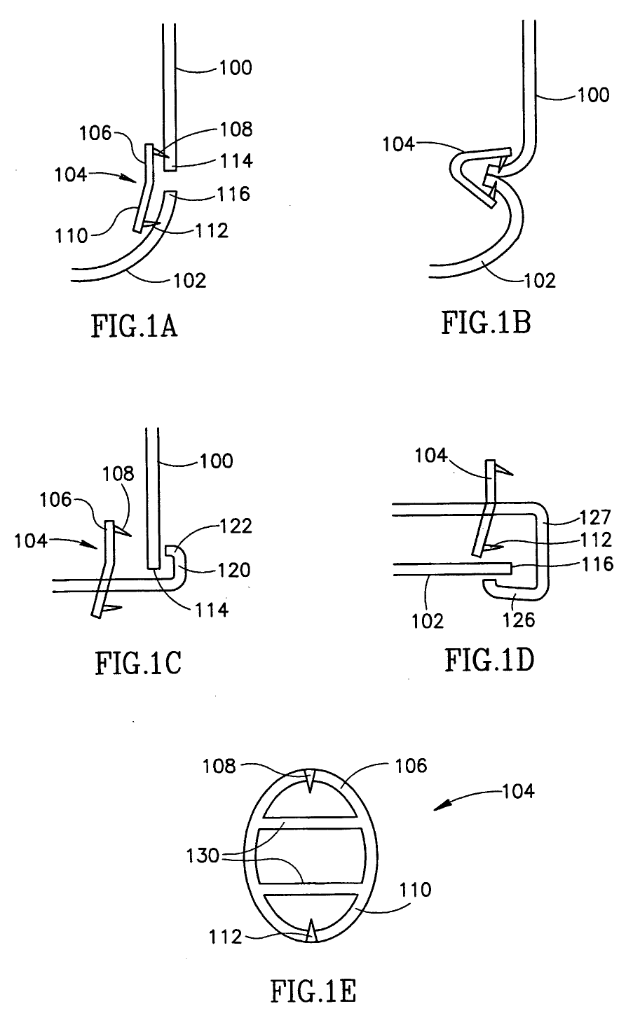

FIGS. 1A-1D illustrate a blood vessel attachment method, in accordance with an exemplary embodiment of the invention. In FIG. 1A, an end-vessel 100 and a side vessel 102 are attached together using a clip 104. Clip 104 comprises a first arm 106 having a vessel engaging means, for example a barb 108, for engaging vessel 100, and a second arm 110, having a vessel engaging means such as a barb 112 for engaging vessel 102. In this and in other embodiments, the blood vessels and/or grafts may be part of the natural vasculature, synthetic, autologus, xenologus, cadaver grafts and/or any other type of blood conduit.

In FIG. 1A the two vessels are engaged by the barbs, such that a lip 114 of vessel 100 is engaged by barb 108 and a lip 116 of vessel 102 is engaged by barb 112. The two lips may abut or there may be a space between them.

In FIG. 1B, clip 104 is folded so the intima portions of the two lips are pressed against each other by the two arms of the clip. The barbs prevent an inadvertent release of the vessel lips during the conformance change and/or provide stability after the connection is completed.

The conformance change of the clip may be effect in various ways, for example elastically, super-elastically or using a shape memory clip, in which cases no external forces may be required. Alternatively, clip 104 is plastically deformed.

Although clip 104 is shown in FIG. 1A having an angle greater than 180° between the arms holding the barbs, in some embodiments, clip 104 is flat or has an angle smaller than 180°, so that the clip can be squeezed shut using a pliers or a clamping scissors. The angle between the arms may affect the ease of mounting the lips of the vessels onto the barbs.

FIGS. 1C and 1D illustrate the use of a puller for retracting the vessel lips onto the barbs. FIG. 1C shows a puller 120 including a curved tip 122 for pulling lip 114 of vessel 100 onto barb 108. FIG. 1D shows a puller 127 including a curved tip 126 for pulling lip 116 of vessel 102 onto barb 112. Alternatively, other methods of mounting may be used, for example manual mounting. An exemplary device for carrying out the method of FIGS. 1C-1D is described below.

In some embodiments of the invention, tip 126 (and 122) is sharp. Alternatively, tip 126 may be blunt, for example to prevent penetration of the tip into the blood vessel wall engaged by the tip. Tip 126 may also be forked, for example, to prevent over penetration of the tip into the blood vessel wall.

Although the above clip is shown for use on side-to-end anastomosis connections, it may also be used for other types of connections, for example, end-to-end connections and side-to-side connections.

FIG. 1E is a top view of a clip suitable for the method illustrated in FIGS. 1A-1D. In this exemplary embodiment, clip 104 comprises an elliptical ring, one side of which is arm 106 and the other side of which is arm 110. The barbs are formed at the apexes of the ellipse. Two cross-bars 130 are provided to allow the clip itself to be used as a pivot for pullers 120 and 127. In an alternative embodiment, clip 140 is bar shaped. As will be described below, a complete anastomosis may require several such clips, or a multi-clip connector.

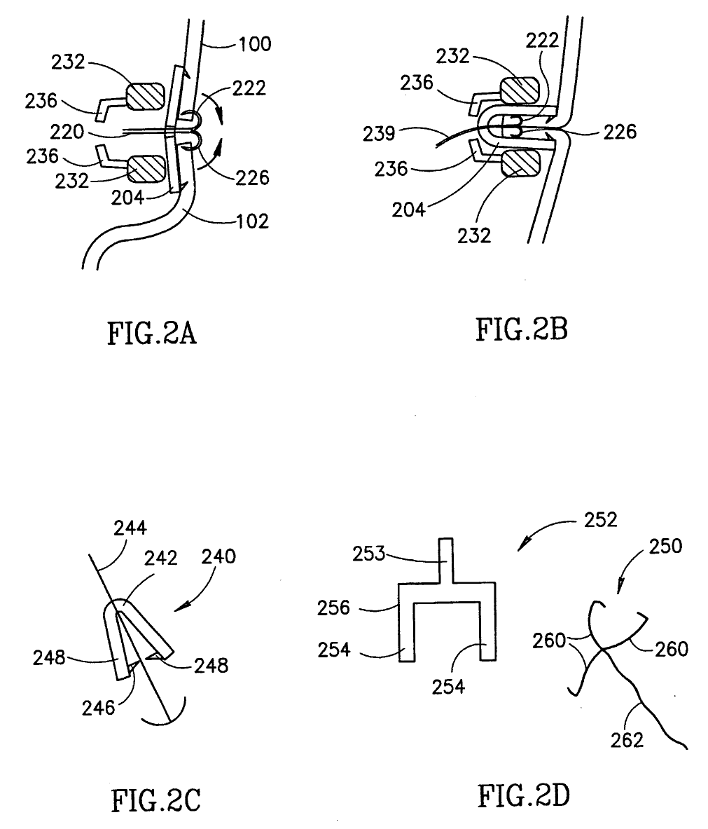

FIGS. 2A-2B illustrate a blood vessel attachment method, in accordance with an alternative exemplary embodiment of the invention. A clip 204, which may be the same as clip 104 of FIG. 1, is mounted on vessel 100 and 102. A single puller 220 including two barbs 222 and 226, one for each vessel is shown instead of two separate pullers. This puller is optionally used for mounting the vessels on clip 104 of FIG. 1. A contra element 232, comprising two spaced apart portions is placed behind clip 104, opposite from barbs 222 and 226. When puller 220 is retracted towards contra element 232, clip 204 is pulled between the contra element portions and bend, thus clamping the two blood vessel lips between the arms. The resulting completed connection is shown in FIG. 2B. Contra element 232 optionally includes an extension 236 on one or both portion, to prevent over retraction of clip 204. Optionally, contra elements 232 are brought closer together, to further seal the anastomosis by bending clip 204. Contra elements 232 may be removed after the procedure is completed such that only clip 204 stays in the body. It should be appreciated similar devices may be used for hole closure, for attaching lips of a same blood vessel.

Puller 220 may be cut at point 234, thus keeping barbs 222 and 226 inside the anastomosis connection. Possibly, the barbs are bio-absorbable. Alternatively or additionally, the barb includes a tissue bonding enhancing material. Alternatively or additionally, the barbs are pulled out of the vessels, possibly tearing the lips of the vessel, at portions outside of the connection area. In one embodiment of the invention, the barbs are shorter than the thickness of the vessel wall, so the tear is not complete. Possibly, barbs 222 and 226 soften at body temperature or in a liquid or electrolytic environment, allowing them to be pulled out. Alternatively barbs 222 and 226 are bent back by bars 130 (FIG. 1E) allowing them to be more easily retracted. Alternatively or additionally, barbs 222 and 226 are not sharp and do not penetrate the vessel wall, but merely pull it back and then can slide past the wall, possibly deforming, and out of the connector. Alternatively or additionally, puller 220 may be connected to the rest of the clip, for example, using a thread or wire.

FIG. 2C illustrates an alternative blood vessel attachment device 240, in accordance with an alternative exemplary embodiment of the invention. Device 240 comprises a clip 242 and a puller 244, generally similar to clip 104 and clip 204. However, clip 242 is pre-bent. Thus, puller 244 is required to pull the tissue lips into a narrow space 246 formed between the two arms of clip 242. Barbs 248 can prevent retraction of the lips, once pulled into space 246. In some embodiments of the invention, space 246 includes a wall (not shown) that divides the space into two spaces, one for each lip. Alternatively or additionally to a wall, a gauze, pad or bioabsorbable material may be provided as a layer between the two lips and/or between the lisp and the device. In an exemplary embodiment of the invention, the layer elutes heparin or other blood coagulation promoters or antagonists, for example, to prevent clots or to promote clotting of leakage blood. Other pharmaceuticals may be provided as well. The pharmaceutical may be soaked in the layer, or, for example, it may be trapped in a matrix, so that it is slowly released over time.

FIG. 2D illustrates an alternative clip 252, in accordance with an exemplary embodiment of the invention. The above described clips, for example that of FIG. 1E have opposing arms. However, this is not required. Clip 252 has one arm 253 on one side of a base 256 and two arms 254 on an opposite side. A matching asymmetric puller 250 is also shown, that has two puller barbs 260 opposing a single puller barb 260, on a thread 262. Arms 253 and 254 may include barbs, as described above. However, in an exemplary embodiment of the invention, the vascular tissue is pinched between the two arms 254, possibly being forced into the space between arms 254 by arm 253. Optionally, the arms extend at an angle, so the space between the arms is more wedge shaped. Possibly, the distance between the arms is shorter than the combined width of the two target blood vessels. Alternatively or additionally, arms 254 can be elastically distorted, to accommodate a greater width of tissue.

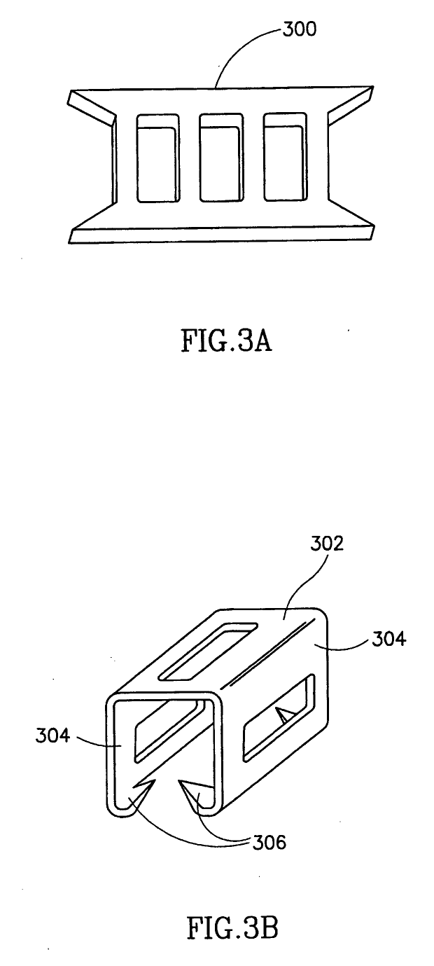

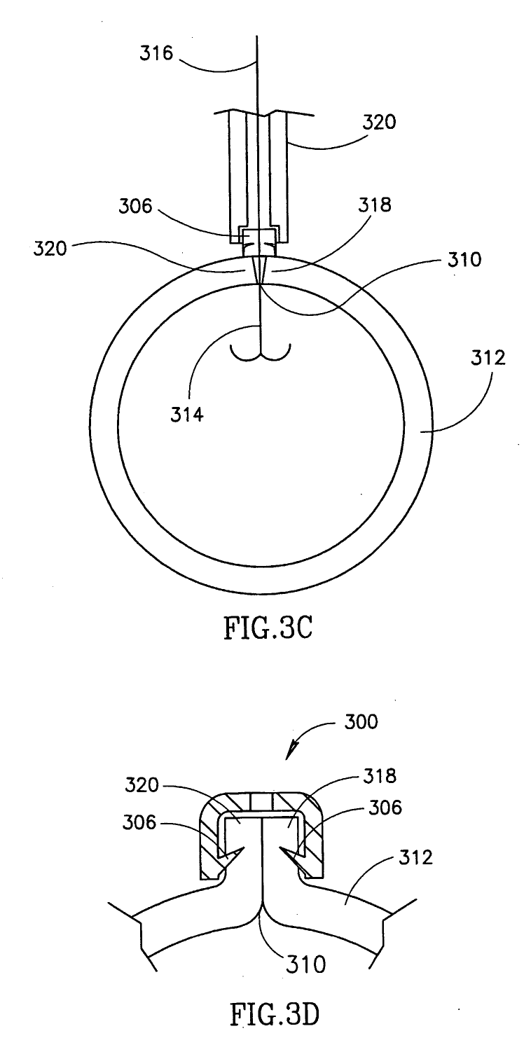

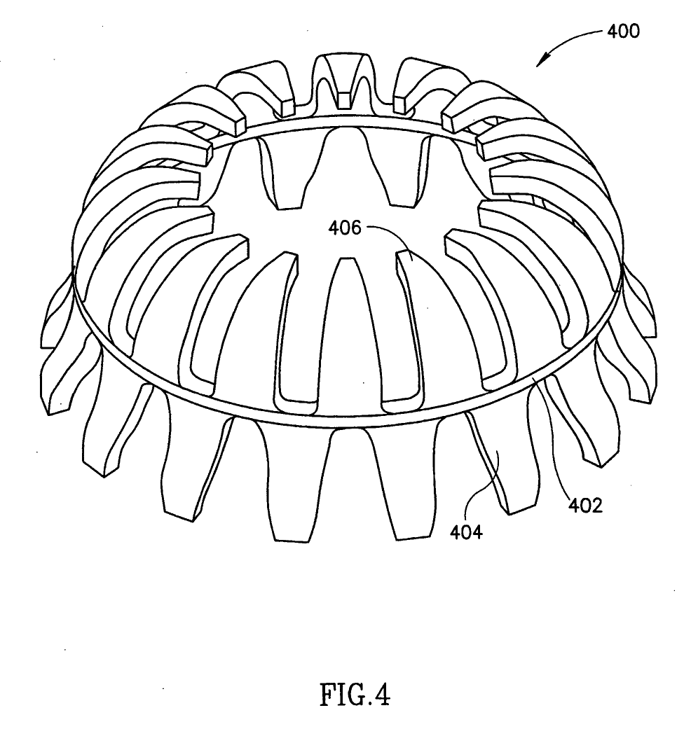

FIGS. 3A-3D illustrate a hole-closure device 300 based on a clip-puller combination, in accordance with an exemplary embodiment of the invention. Although FIGS. 1 and 2 are described with reference to attaching two lips from different blood vessels, similar principles may be applied to vascular hole closure, in which the two lips that are sealed together are from a same blood vessel.

FIG. 3A shows a hole closure device 300, in a layout view. In FIG. 3B, device 300 is folded, such that it has a base 302 and two arms 304, each arm having one or more barbs 306 at its ends.

FIG. 3C shows a device 300 being deployed. In an exemplary embodiment of the invention, device 300 is held against a vessel 312, adjacent a hole 310 therein, by a contra-tube 320, which may be hollow. Alternatively, device 300 may be mounted on tube 320. Hole 310 has lips 318 and 320, that can be pulled into device 300, by a puller 314. Possibly, puller 314 is pulled using a thread or wire 316, which optionally extends through tube 320.

FIG. 3D shows hole 310 after puller 314 pulls lips 318 and 320 into device 300. Puller 314 (which may remain in the connection) is not shown, for example being retracted (possibly through the hole in base 302, optionally after being bent back by contact with base 302 itself) or being absorbed, as described above. Device 300 may be sized to fit various hole sizes and blood vessel thickness. Alternatively or additionally, a flat device, for example as shown in FIG. 3A is bent, on the spot, to match desired hole closure characteristics. Although device 300 is shown with two barbs 306 on each arm, a greater or smaller number of barbs may be provided, possibly not opposing barbs, or even protrusions (not sharp) instead of barbs. The length of the device may depend, for example on the size of hole 310. Alternatively or additionally, a plurality of devices 300 are applied size by side.

Similarly, the clips of FIGS. 1-2 may be provided as individual clips, for example, one by one or using a multi-clip delivery system, for example for simultaneous delivery. Typically, several clips are required for connecting two blood vessels together, for example, 3, 4, 5, 6 or more clips. Alternatively, the clips may be connected together, for example using a thread or a ring, to form a single anastomosis connector (or hole closure device). Alternatively, previously described connectors and hole closure devices (e.g., in the above PCT applications) may be segmented to provide clips, for example each clip comprising two opposing spikes and an optional ring section.

Alternatively or additionally to bending of the spikes/arms, a torsion bar mechanism may be provided for rotation of the arms. In an exemplary embodiment of the invention, base 256 of clip 252 in FIG. 2D can serve as a torsion bar, that twists alternatively or additionally to bending of arms 254 and 253. Clip 252 can then be a pressure based clip, which simply forcefully contacts two vascular tissues.

FIG. 4 illustrates a multi-clip connector 400, in accordance with an exemplary embodiment of the invention. Connector 400 comprises a ring 402 having a plurality of “side” engaging clips arms 404 and an opposing plurality of “end” engaging clip arms 406. As shown, opposing clips arms do not need to have a same radial position, however, that is possible. Alternatively or additionally, the number of “side” and “end” clip arms may be different. Alternatively or additionally, the connector may be used for two “side” vessel or for two “end” vessels. As shown the clip arms are not designed to penetrate the vessel walls, however, in some embodiments, at least some of the clip arms may penetrate the vessel walls, such arms may include a fork design or a protrusion distal from their tip, to prevent over penetration and/or motion of the vessel wall along the arm. The clip arms on opposing sides may have the same or a different general design. Alternatively or additionally, the clip arms on a same side of ring 402 may also be the same (as shown) or different, for example alternating clip arms having different designs.

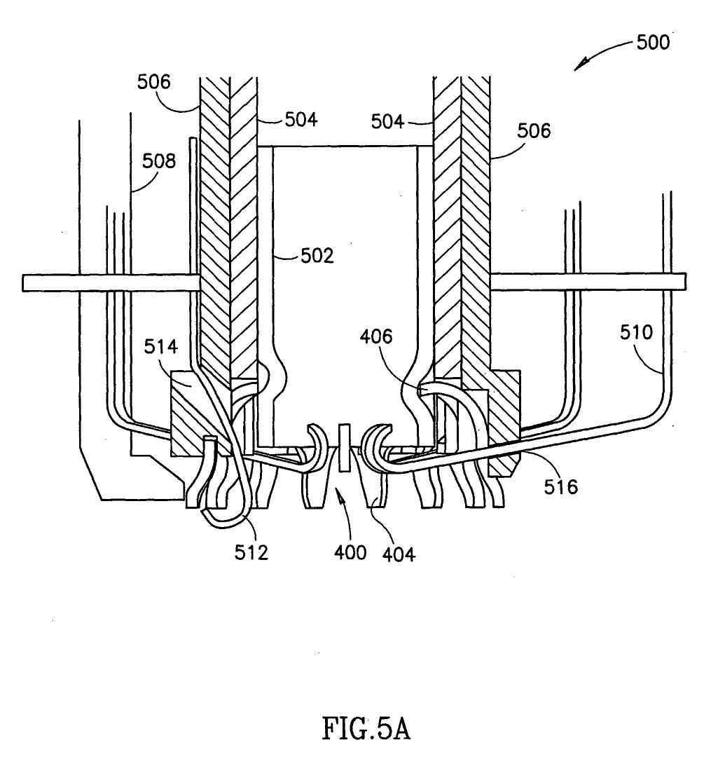

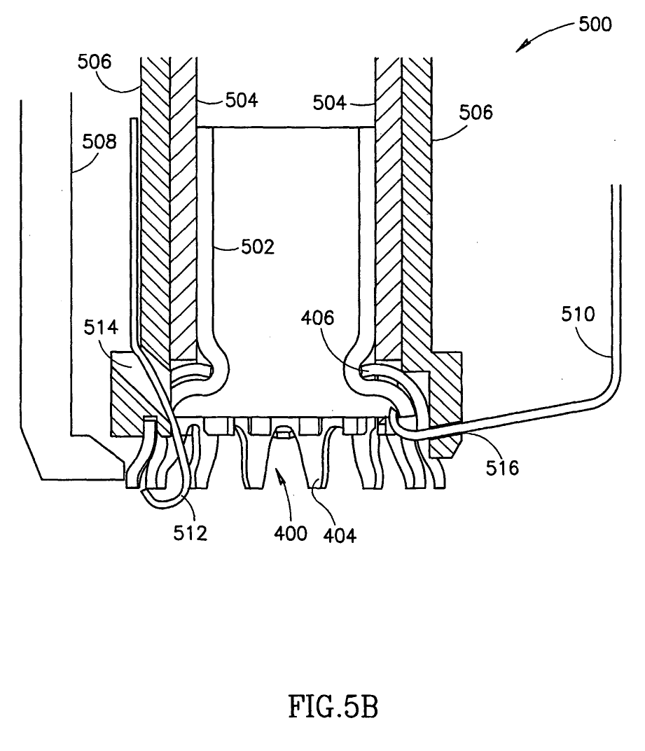

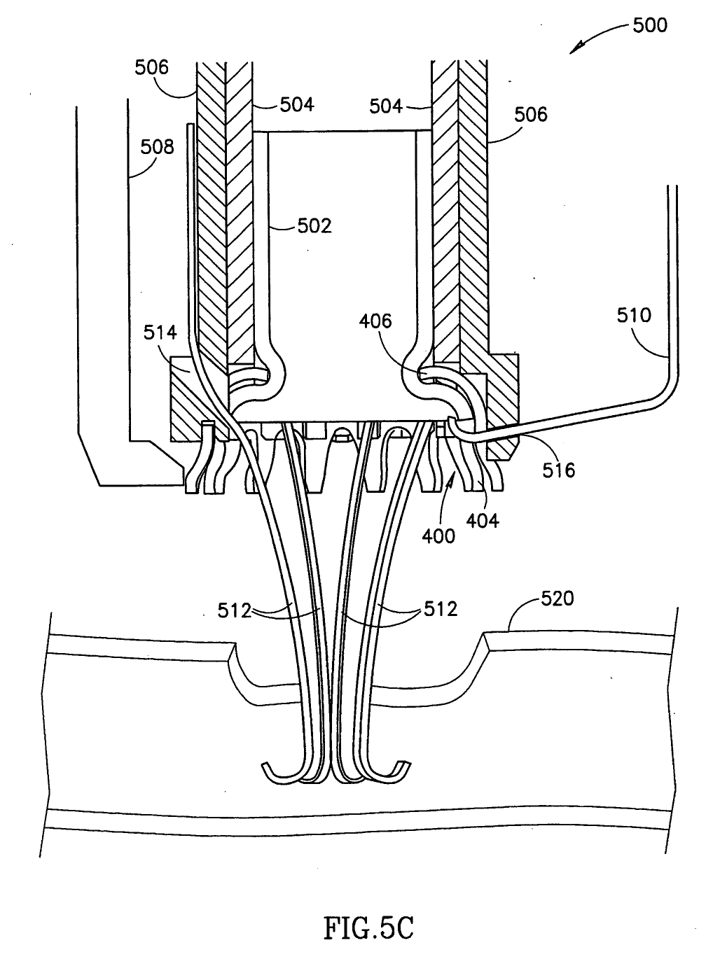

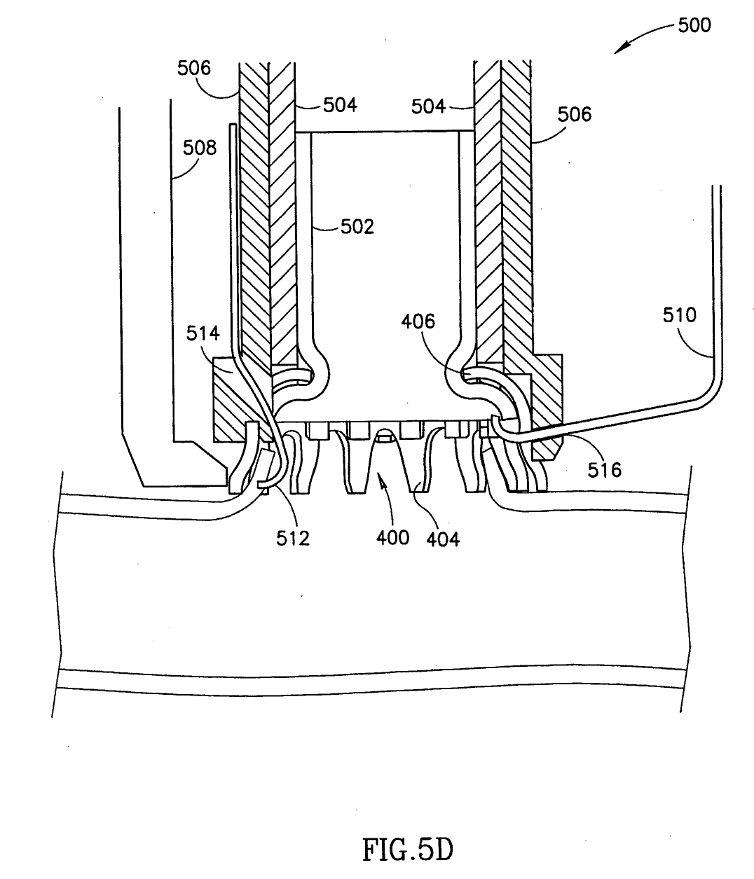

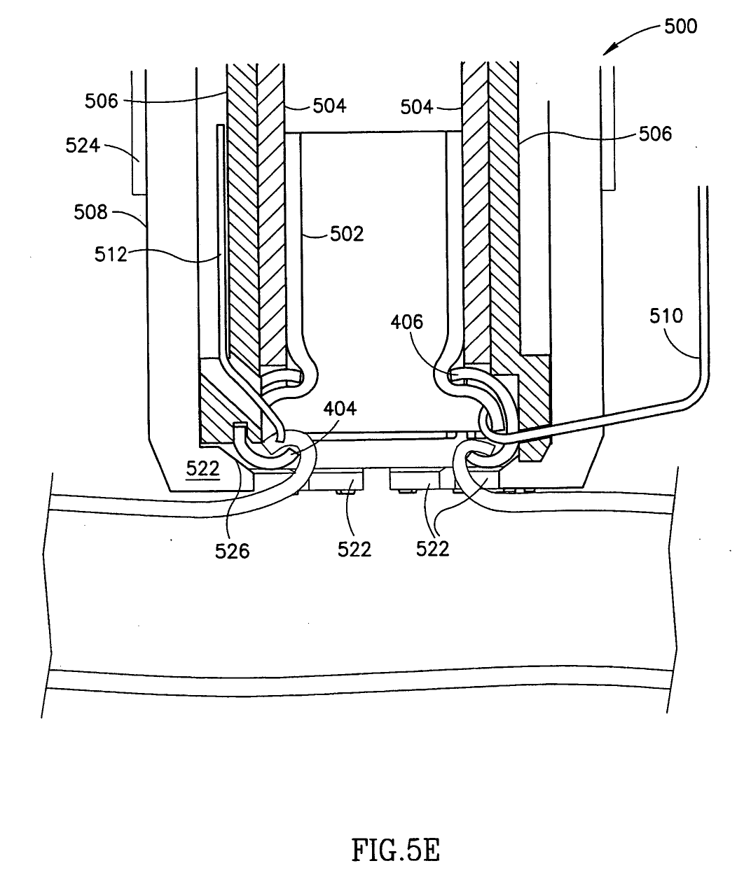

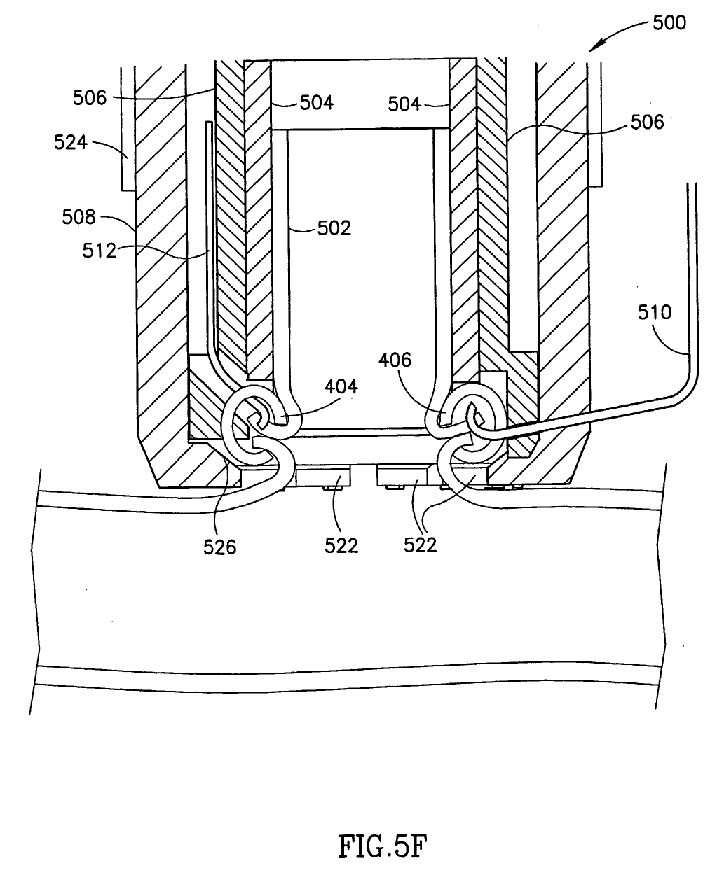

FIGS. 5A-5F illustrate a method of deploying the clip of FIG. 4, in accordance with an exemplary embodiment of the invention. In these figures, not all the repeating elements are shown, to reduce visual clutter. System 500 may be deployed, for example in open surgery, in endoscopic or throactoscopic surgery and/or in a transvascular approach.

FIG. 5A illustrates a delivery system 500 having mounted therein a graft 502 and a connector 400. The view is a cross-sectional view selected so that the operation of one “side” arm 404 and one “end” arm 406 are clearly visible.

System 500 includes an outer contra tube 506 and an inner pusher tube 504. Connector 400 is held, for example elastically or by friction, by outer contra tube 506. An outer base tube 508 is provided for closing the “side” arms, as will be described below. A plurality of “side” vessel pullers 512 are provided through apertures 514 formed in outer tube 506. A plurality of “end” vessel pullers 510 are provided through apertures 516 formed in outer tube 506. In an exemplary embodiment of the invention, arms 564 and 506 are staggered, so that apertures 514 and 516 are staggered to match the arm locations. The tips of the pullers may or may not be aligned with the arms into which they pull vascular tissue. In some embodiments, at least some of the pullers may be non-planar.

In FIG. 5B, “end” pullers 510 are retracted, pulling the lips of graft 502 against clip device 400. This step may be performed inside the body or outside of it.

FIG. 5C shows system 500 near a target side vessel 520. Target vessel 520 may be, for example, a coronary artery, a synthetic or biological graft, an aorta, a LIMA, a coronary vein, an aorta or a peripheral blood vessel, such as a femoral artery or a leg vein, or any other known blood conduit. Also graft 502 may be any known blood conduit. Pullers 512 are extended forward so that they enter an opening in vessel 520. In some embodiments of the invention, the insertion of pullers 512 is manual. In others, it is facilitated by an alignment of system 500 and the hole in the target vessel. It is noted that a punch for forming the opening may be provided through outer base tube 508 and then replaced with the graft delivery portion. Alternatively, a punch may be provided through graft 502. Alternatively, a separate punching tool is used. Alternatively, an incision is made using a knife.

In FIG. 5D, pullers 512 are retracted, pulling the lips of the incision of vessel 520 into the clip, so that the lips of the two vessels are near, touching or overlapping each other.

In FIG. 5E, a proximal inwards pointing portion 522 of base 508 is pushed inwards, causing arms 404 to close on the lips of vessel 520, and in the process possibly also everting them further. Portions 522 may include an inclined portion 526 for guiding the arms to close in a desired fashion. One exemplary method of moving portions 522 is advancing an outside tube 524 over base outer tube 508, causing it to radially compress. Although puller 512 may be distorted during the closing of arms 404, this is generally of no consequence.

In FIG. 5F, inner pusher tube 504 is advanced, closing arms 406 of clip 404. Base tube 508 may serve as a contra for the pressure. The blood vessels are thus held securely between arms 404 and 406, preferably preventing blood leakage.

In some embodiments of the invention, the steps of FIGS. 5E and 5F are performed simultaneously or in an a opposite order. Although simultaneous performance for all the arms on a side is preferred, in some embodiments, not all the arms on a single side are closed together.

The anastomosis being completed, pullers 512 and 510 may be retracted and base tube 508 can be radially expanded, to release clip 400. Inner tube 504 may be advanced further to release clip 400 from outer tube 506. Alternatively, the pullers may be dealt with as described above, for example, cut and left in the body, possibly to be absorbed.

The above, described the deployment of a plastically deployed device. In an elastic, shape-memory or super-elastic device, a similar delivery system may be used. For example, base tube 508 may include barbs or an inner lip for maintaining arms 404 open (until base tube 508 is advanced) and inner tube 504 may include an extension for preventing arms 406 from closing (until inner tube 504 is retracted). Even in a plastically deformed device 400, ring 402 may be elastic, for example to allow radial compression for deployment and/or for being held by outer tube 506.

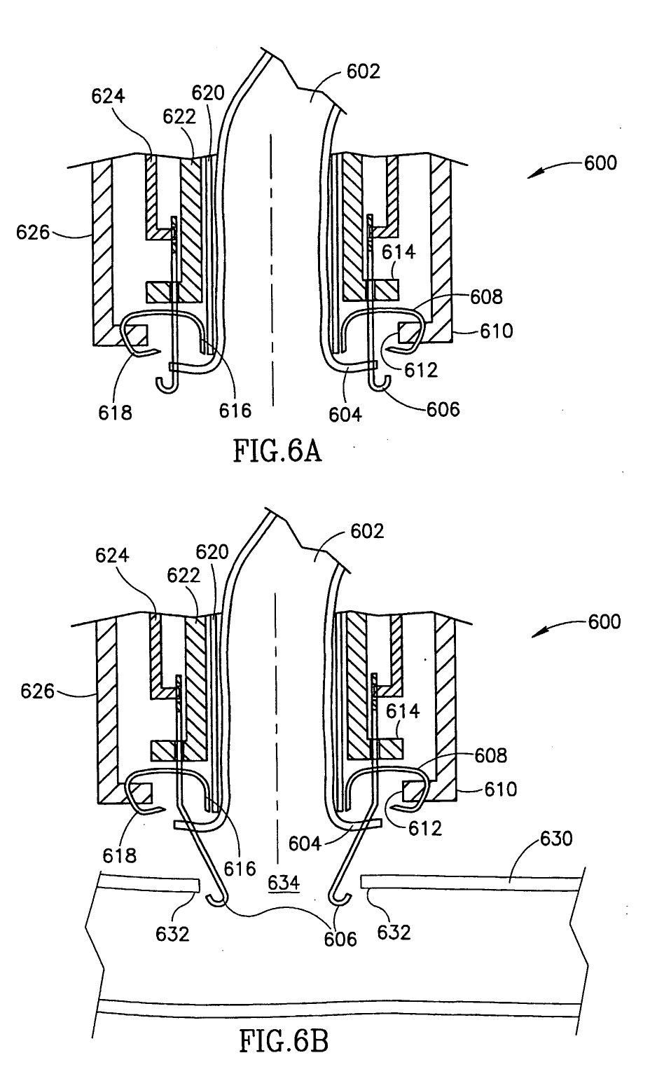

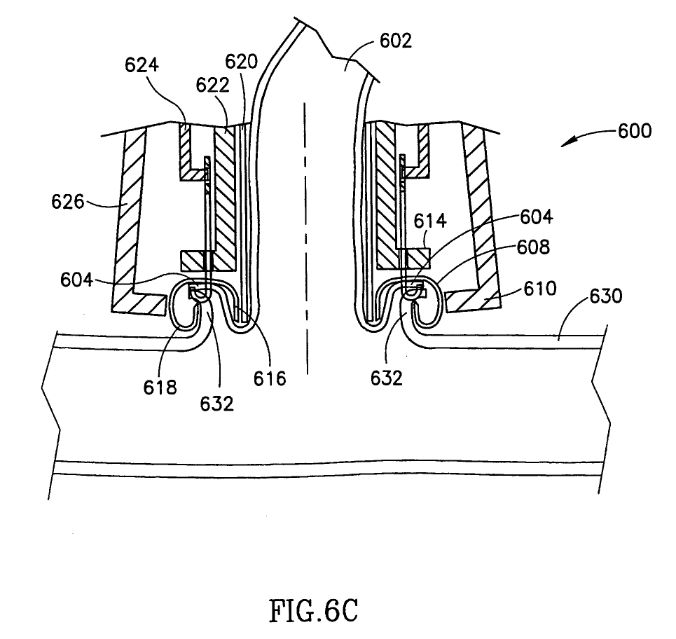

Although FIG. 5 above and FIG. 6 below describe a side-to-end anastomosis, it should be appreciated that a similar mechanism may be used for oblique, side-to-side and end-to-end connections. In such connections, the vessel may be aligned in a non-axial manned to the rest of delivery system 500, for example, be provided through a lumen that is perpendicular to the system axis. However, the general working of the pullers remains the same.

Alternatively or additionally, system 500 is a split system (into two, three or more lengthwise parts), so that it can be more easily removed from graft 502.

FIGS. 6A-6C illustrate an alternative method of deploying multiple clips in an anastomotic connection, in accordance with an exemplary embodiment of the invention.

FIG. 6A shows a graft 602, having a lip 604 transfixed on a puller 606. A delivery system 600, mounted on the graft includes an inner tube 620 on which an anastomotic connector 608 is mounted. Connector 608 may comprise, for example, a ring 616 and a plurality of clip arms 618. In an exemplary embodiment of the invention, connector 608 is super-elastic, elastic or shape memory, with arms 618 prevented from folding in by a restraint 626. In an exemplary embodiment of the invention, an inwardly pointing extension 610 of restraint 626 includes a circumferencially pointed (e.g., out of the figure plane) bump 612 (or restraint 626 is slotted) that prevents arm 618 from radial motion. When restraint 626 is rotated relative to connector 608 or when restraint 626 is radially expanded, arms 618 are released.

Delivery system 600 further comprises a retracting tube 624 for retracting pullers 606 and a contra tube 622, having an optional lip 614, which prevents retraction of connector 608.

In FIG. 6B, pullers 606 are extended into an incision 634, having lips 632 in a “side” vessel 630.

In FIG. 6C, pullers 606 are retracted, everting lips 632 and pulling both lips 632 and lips 604 into connector 608. Restraint 626 then releases arms 618, allowing the connector to close, sealing the connection between graft 602 and vessel 630. Pullers 606 are thus generally not required any more, at least not for holding vessel 630. Pullers 606 can then be further retracted, possibly causing no damage to the blood vessels, as the pullers are straightened by the retraction. Connector 608 can be released, for example by advancing contra tube 622. In a plastically deformed embodiment, restraint 626 acts as a anvil, to radially compress arms 618.

Arms 618 may have sharp tips, as shown, for example to penetrate one or both of lips 632 and 604. Alternatively, the tips of arms 618 may be blunt, to apply non-penetrating pressure. Alternatively, arms 618 may hold lips 604 against the upper part of the connector and lips 632 against the ring part of the connector.

It should be noted that while FIGS. 5 and 6 illustrate anastomotic connectors, a similar delivery system may be used for connecting two lips of a single blood vessel, for example for hole closure. In such a case, the connector, instead of being a ring as shown in FIG. 4, may be a line connector or a circular connector with arms only on its bottom part, pointing in.

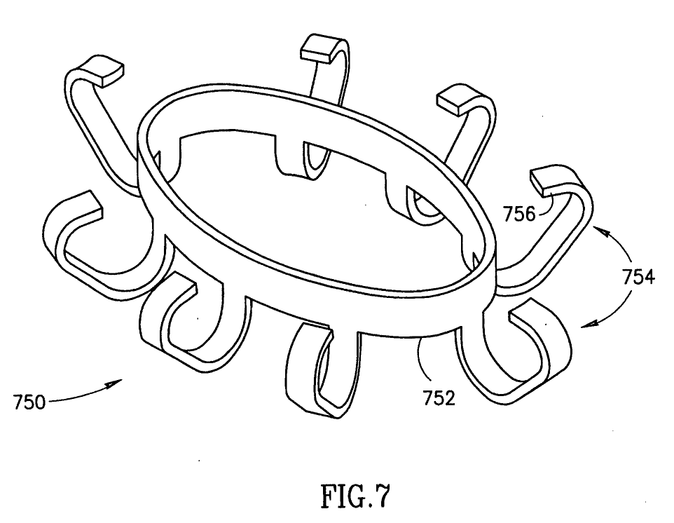

FIG. 7 is a ring-clip anastomosis connector 750, in accordance with an alternative exemplary embodiment of the invention connector 750 comprises a ring 752, on which a graft is optionally everted and a plurality of fingers 754 which are curved, for example, In a “C” shape as shown, so that they can seal a blood vessel lip against the everted graft. In an optional embodiment, ring 752 is flexible and/or absorbable, for example, being made of a suture or plastic. Alternatively, ring 752 and fingers 754 formed of a single contiguous element of a single material. Optionally, fingers 754 have blunt tips 756, which tips do not pierce the blood vessels being held.

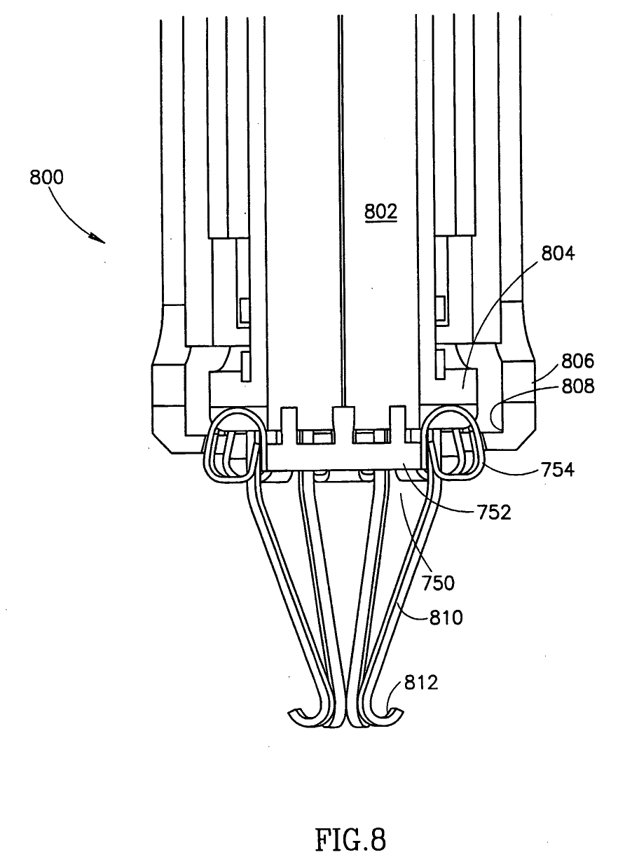

FIG. 8 is a cut-through view of a tip of a loaded delivery system 800 for delivering connector 750, in accordance with an exemplary embodiment of the invention.

Delivery system 800 comprises a retractable inner tube 802, a base tube 804 against which connector 750 is maintained and an outer tube 806. In an exemplary embodiment of the invention, tube 802 is used for retracting- and/or is contiguous with a plurality of pullers 810. Optionally, pullers 810 end at a hook 812, adapted to engage the lips of an opening of a target vessel. Optionally, pullers 810 are arranged as a cone and are outside of ring 752. Thus, when retracted, the pullers tend to extend out radially.

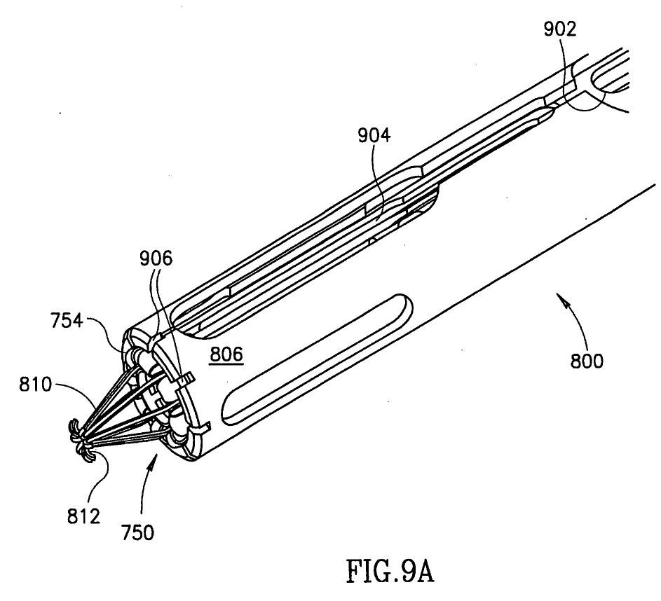

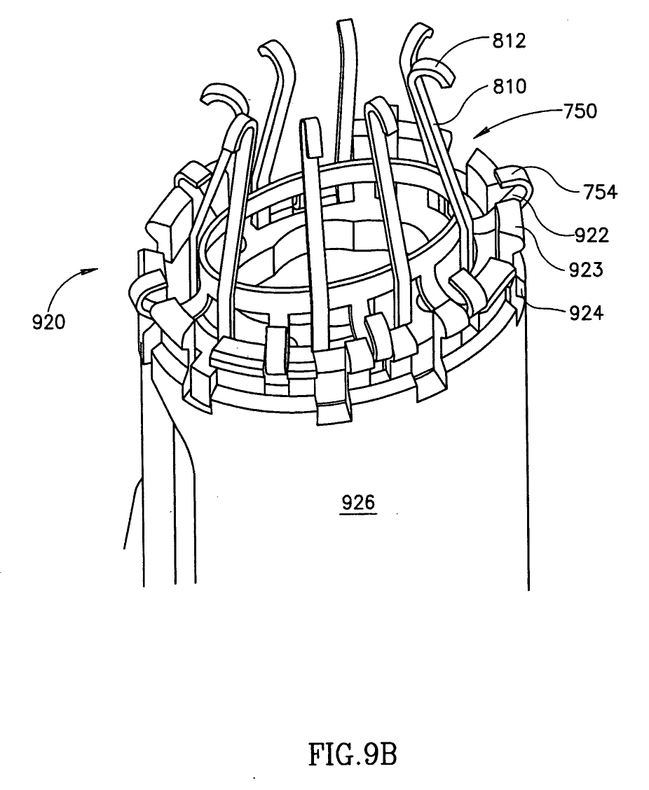

In an exemplary embodiment of the invention, outer tube 806 defines an inner step 808, into which fingers 754 may be pulled and restrained, as described in FIG. 9A.

FIG. 9A is a perspective view of the tip of the delivery system 800. Outer tube 806 defines a plurality of slots 906, each corresponding to a finger 754 of connector 750. In an exemplary embodiment of the invention, a thin object, such as a pen or a nail is used to pull the finger behind step 808 (FIG. 8), using slot 906 as a guide. For example, the pen is placed inwards of the finger and guided by the slot is pulled back and out, pulling the finger back with it. In an exemplary embodiment of the invention, connector 750 is elastically (or super-elastically) deformed by this manipulation, so that when outer tube 806 is retracted, relative to base tube 804, the fingers snap back to their resting position (shown in FIG. 8) and engage vascular tissue between fingers 754 and ring 752 or within a finger.

Alternatively, connector 750 may be plastically deformable. For example, the advance of outer tube 806 may close the fingers against the ring. The resting position, may thus have slightly open fingers. Also in an elastic device, a slight gap may be desirable, for example, to prevent pinching of the vascular tissue by the fingers.

In an exemplary embodiment of the invention, a graft (not shown) is provided through an opening 902 in delivery system 800 and optionally everted over ring 752. PCT application PCT/IL01/00069 describes an exemplary method of pulling a graft through a delivery system and PCT application PCT/IL01/00074 describes exemplary methods of everting the graft.

The graft may be everted over ring 752 before or after fingers 754 are pulled back.

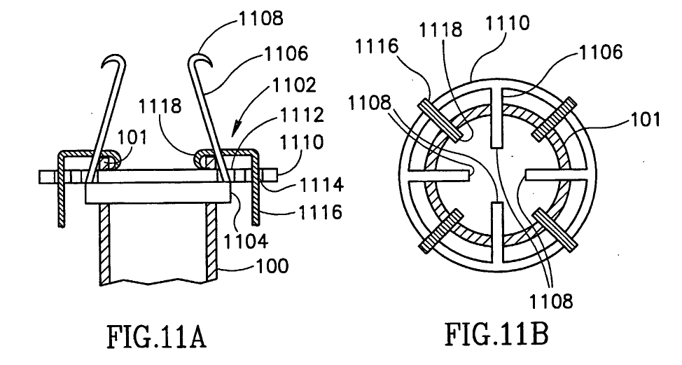

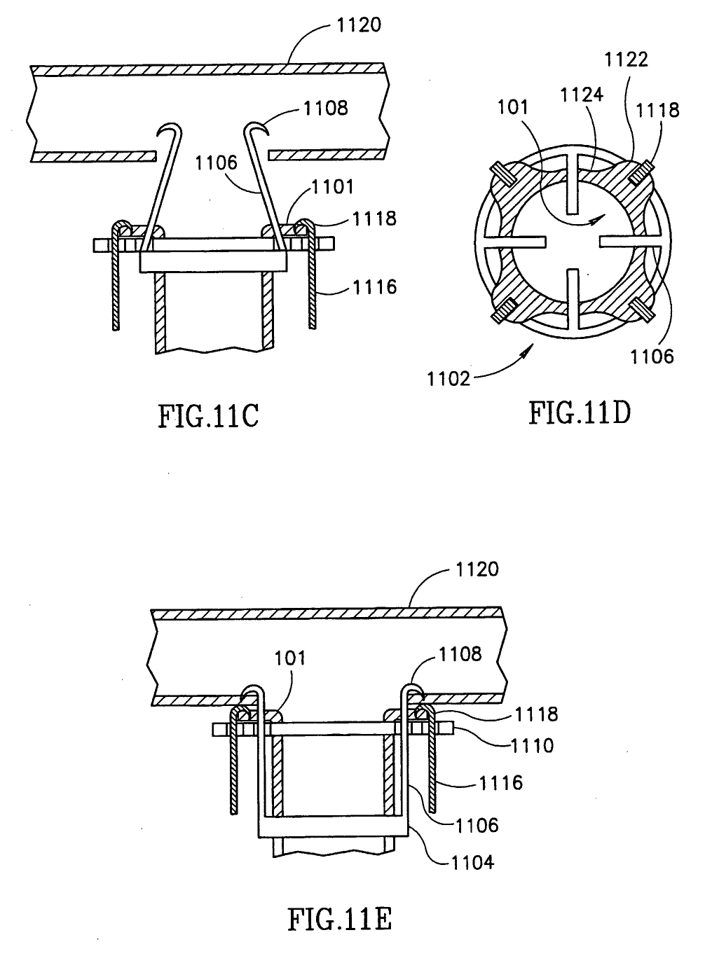

Optionally, pullers 810 transfix the graft. In one example, fingers 754 are pulled back, the graft is everted over ring 752 and then the pullers are advanced to penetrate the graft. Alternatively, for example as described below in FIG. 11, the pullers do not pierce the graft. Instead, the (at least partially) everted graft is held in place by some or all of the fingers, in closed position. The rest of the fingers may be pulled back, to be released by outer tube 806.

In an exemplary embodiment of the invention, when pullers 810 are retracted, they pull back the lips of the target vessel, adjacent ring 752, so that when outer tube 806 is retracted, fingers 754 are released to engage the lips. Alternatively, pullers 810 pull the lips between already closed fingers 754 and ring 752.

In an exemplary embodiment of the invention, delivery system 800 is formed with a pre-formed split 904, so that when the connection is completed, system 800 can be split and easily removed from the graft. In an exemplary embodiment of the invention, tube 802 includes a knife or extension that causes system 800 to split, when retracted. Tube 802 itself may be pre-split.

FIG. 9B is a perspective view of the tip of an alternative loaded delivery system 920, in accordance with an exemplary embodiment of the invention. An outer tube 926 defines a plurality of slots 922 each wide enough to contain a finger 754. In an exemplary embodiment of the invention, the slots are slightly narrower (e.g., between 1% and 20%) than a finger 754, so that when a finger is pulled into the slot, it twists a small amount and is maintained in place by an outer lip 923 of outer tube 926. Retraction of outer tube 926 will cause the fingers to distort and then be released back to their resting position. Optionally, the fingers widen at the point where they meet the slot.

Also shown are a plurality of optional slots 924 situated between slots 922, which may be used for penetration of the graft by pullers 810. In an exemplary embodiment of the invention, the graft is everted over lip 923 and then pullers 810 are advanced. Slots 924 are used to guide a narrow object that forces the graft onto the sharp end of hooks 812, so that the hooks penetrate the graft. Alternatively or additionally, slots 924 are used to guide the straightening of pullers 810 when they are retracted out of the blood vessels.

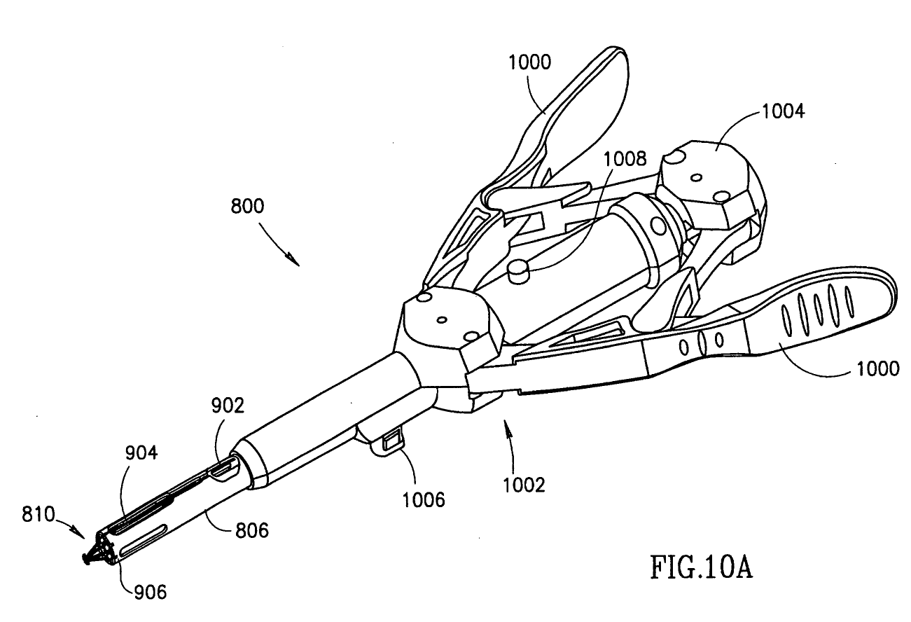

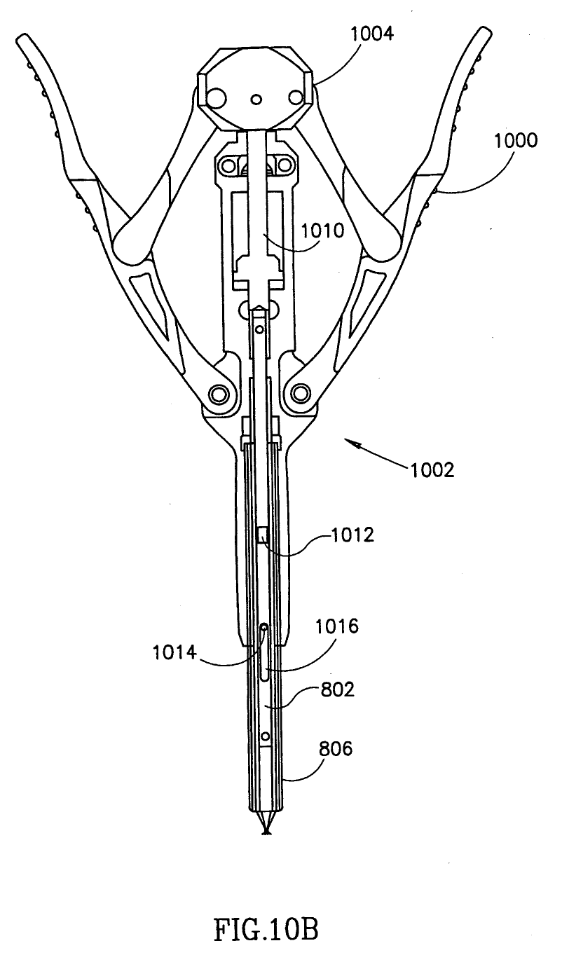

FIG. 10A is a perspective view of the complete loaded delivery system 800, having a body 1002. In an exemplary embodiment of the invention, inner tube 80 is retracted by squeezing a pair of levers 1000, so that a base 1004 of tube 802 is retracted. One or more safety pins 1006 and 1008 may be provided, for example, to prevent inadvertent retraction of tube 1008 and/or to control the progression of operations steps. In an exemplary embodiment of the invention, when inner tube 802 is sufficiently retracted, its motion is coupled to a retraction (or advance) of outer tube 806, so that the fingers 754 are released.

FIG. 10B is cut, through a side view of the complete loaded delivery system 800. Base 1004 is shown coupled to a shaft 1010 which may be attached, coupled or contiguous with inner tube 802. Optionally, a narrowing 1012 is provided in tube 802, to match with a safety pin, such as pin 1006. In an exemplary embodiment of the invention, a pin 1014 of fixed to outer tube 806 interacts with a slot 1016 of inner tube 802, to allow inner tube 802 to retract outer tube 806, once inner tube 802 is sufficiently retracted.

FIGS. 1A-11E illustrate a connector 1102 in which a partial eversion is achieved, in accordance with an exemplary embodiment of the invention. Connector 1102, is superficially similar to connector 102, in that it has a ring 1104 on which a plurality of spikes 1106 having hook tips 1108 are mounted. These spikes pass through apertures 1112 in a base ring 1110. In one embodiment of the invention, however, base ring 1110 includes a second array of apertures 1114, through which a plurality of graft-pulling spikes 1116, having hooked tips 1118, are provided. These spikes may be mounted on a second ring (not shown) or they may be part of the delivery system.

In this connector, instead of everting graft 100 over spikes 1106, graft end 101 is distorted so that it is at least partially everted over base ring 1110, but abuts the spikes instead of being transfixed by them.

FIG. 11A shows a starting position, in which graft 100 is inserted into connector 1102, and puller spikes 1116 are bent over so that hooks 1118 are positioned to radially distort graft end 101.

FIG. 11B shows a top view of FIG. 11A.

FIG. 11C, shows the effect of pulling spikes 1116, so that hooks 1118 engage and pull back graft end 101. Spike hooks 1108 are shown in position inside a target vessel 1120.

FIG. 11D is a top view of connector 1102 in FIG. 11C, showing that portions 1122 of graft end 101, which are between spikes are pulled past spikes 1106. Portions 1124 that are adjacent spikes are pulled back to abut spikes 1108. In general, both types of portions are everted 90°, so that their intima can contact target vessel 1120. Optionally, a radial depression is formed in the base of spikes 1106, to allow portions 1124 to be pulled out more.

In FIG. 11E, spikes 1106 are pulled back (e.g., by pulling back ring 1104), so that hooks 1108 engage target vessel 1120 and the anastomosis is completed.

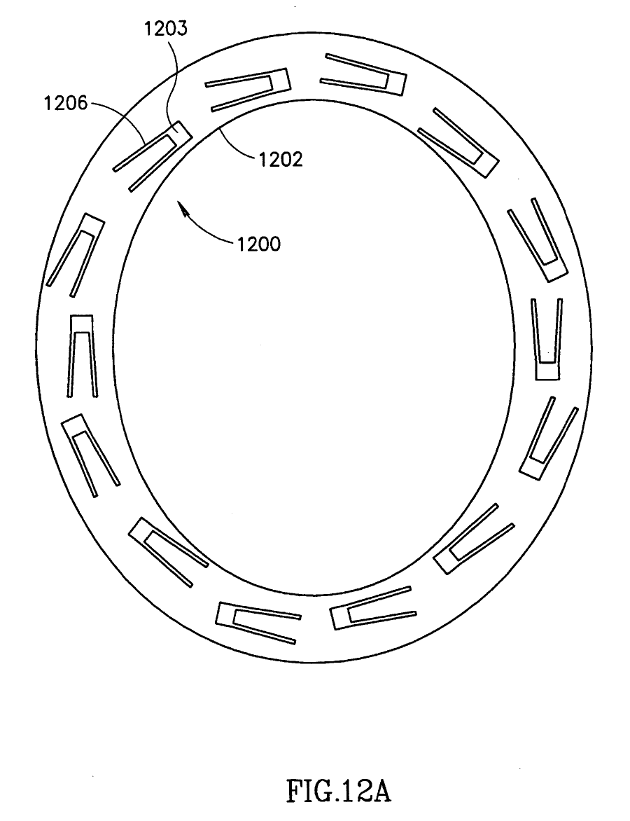

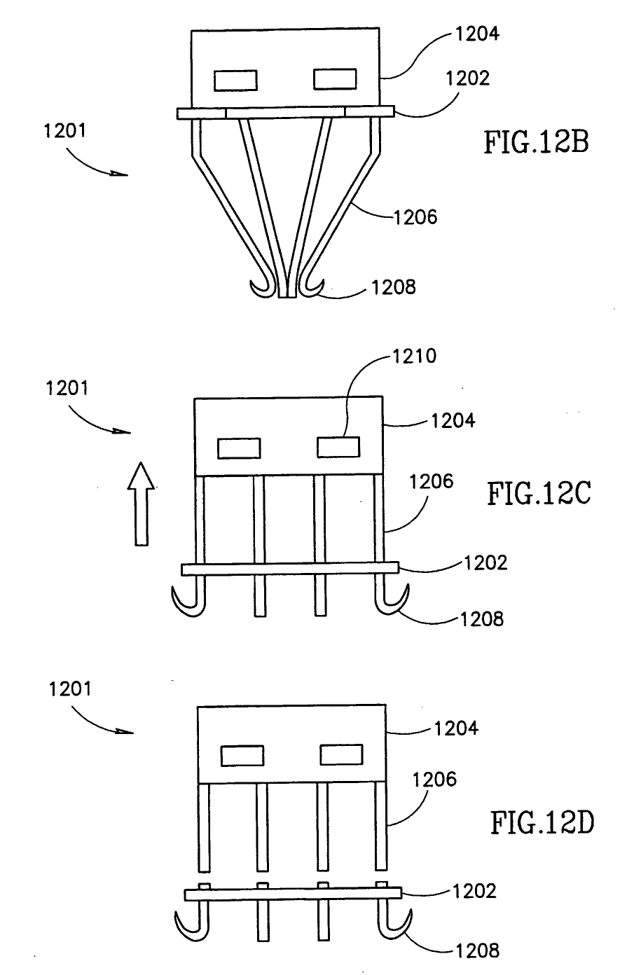

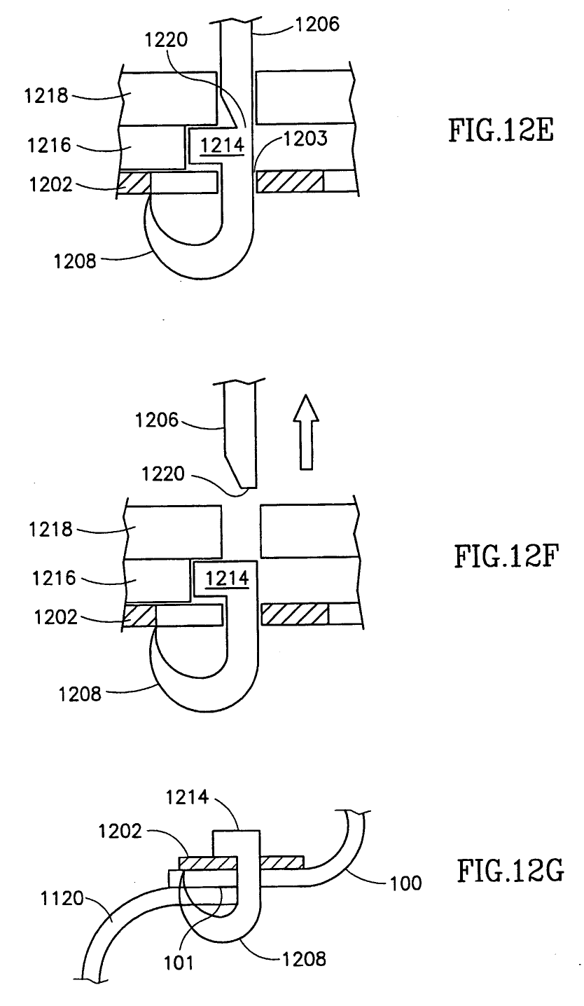

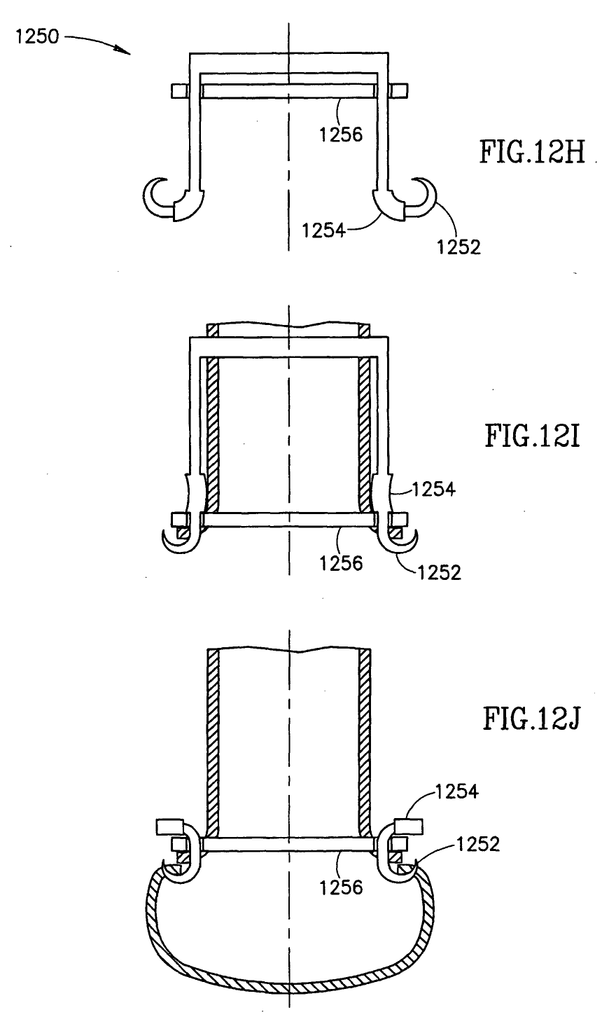

Optionally, spikes 1116 are further retracted, so that they release graft end 101 and are removed from the body. In some embodiments, spike hooks 1118 may rip through portions 1124. Alternatively or additionally, spikes 1116 are made of a bio-absorbable material. Possibly, spikes 1116 are attached to a delivery system used to deliver and deploy connector 1102 and graft 100. Alternatively, spikes 1116 are cut, so that hooks 1118 remain in the body. Alternatively, for example as shown in FIG. 12, parts of spikes 1116 are torn off.

As shown, apertures 1114 are further out radially than apertures 1112. However, they may be at a same radial distance in other designs.

In an alternative embodiment of the invention, apertures 1114 are formed in a separate ring (not shown), which is part of the delivery system (not shown). After deployment, this other ring may be removed from the body.

Alternatively or additionally to apertures 1114 and 1112 being enclosed apertures, slots or slits in ring 1110 (e.g., with openings to the outside of ring 1110) may be provided instead.

FIG. 12A illustrates an exemplary base ring 1200 of an anastomotic connector, in accordance with an exemplary embodiment of the invention. Ring 1200 may be used for any of the connectors described above. Ring 1200 includes a base part 1202 having formed therein a plurality of apertures 1203 for allowing spikes to pass through. Optionally, each aperture includes a leaf-spring section 1206. Possibly, when a hook is pushed through aperture 1203, the hook pushes the leaf-spring aside. In an alternative embodiment, apertures 1203 are defined as slots on the outside and/or inside of base 1202.

FIGS. 12B-12D illustrate a process of deploying a connector in which part of the connector is removed, in accordance with an exemplary embodiment of the invention. FIG. 12B shows a connector 1201 having a base ring 1202, for example as in FIG. 12A and a plurality of spikes 1206, having hook-tips 1208, mounted on a ring 1204.

In use, after graft 100 is mounted on spikes 1206, for example using one of the methods described above, hooks 1208 are placed into a target blood vessel, such as vessel 1120 (FIG. 11C). Ring 1204 is then retracted (FIG. 12C), for example by engaging a plurality of apertures 1210 formed therein, so that spikes 1206 and hooks 1208 are retracted and seal the anastomosis (Se also FIGS. 11A-11E). In FIG. 12D, ring 1204 and most of the length of spikes 1206 is cut off of hooks 1208. Optionally, spikes 1206 are torn, at a location that is pre-weakened for such tearing. Such weakening can be, for example, by thinning or holing the connector or by chemical and/or heat treatment. In an exemplary embodiment of the invention, the weakening is formed at a distance that allows the connector to connect two vessels and, optionally, means for locking the hook portion to the ring.

FIGS. 12E-12G illustrate the effect of the process of FIGS. 12B-12D, on a single spike of the connector. FIG. 12E shows a spike 1206 that includes a weakening 1220. Optionally, spike 1206 includes an extension 1214. In an exemplary embodiment of the invention, extension 1214 is used to prevent spike 1206 from falling off ring 1202, through aperture 1203. Alternatively or additionally, extension 1214 prevents retraction of hook 1208 while tearing spike 1206. Optionally, such an extension is defined on only some of spikes 1206.

In an exemplary embodiment of the invention, a stopper 1218, for example a ring, is provided to prevent hooks 1208 from retracting during the tearing. Such a stopper may be urged against extension 1214. Alternatively or additionally, the stopper may engage the spike, for example, by clamping on it. An optional spacer 1216 may be provided to couple stopper 1218 to ring 1202. Optionally, the clamping crimps and/or partially cuts spike 1206, so that the weakening is caused or exacerbated by the crimping.

In an exemplary embodiment of the invention, ring 1202 is an ellipse. In an exemplary embodiment of the invention, the graft everted unevenly or is cut at an angle, so that when the anastomosis is complete, an oblique connection will form. In an exemplary embodiment of the invention, the axis of the ellipse is use to select the inclination direction of the connection. Such an elliptical ring may also be used in the other embodiments herein.

In FIG. 12F, spike 1206 is retracted, while extension 1214 is held, so spike 1206 is tom at weakening 1220.

FIG. 12G, shows the final completed anastomosis between graft 100 and target vessel 1120 (for a single hook 1208).

Alternatively or additionally to providing an extension 1214, spikes 1206 may be pre-stressed (e.g., be super-elastic or have shape memory), so that the torn part of the spike folds back over to fold back over ring 1202. Alternatively or additionally, the end of the spike is bent over.

FIGS. 12H-12J illustrate a connector 1250 with self-curling spikes, in accordance with an exemplary embodiment of the invention. In connector 1250, a plurality of forward spikes 1252 are curled, at least at their tips. However, when spikes 1252 are retracted through a base ring 1256, their tips are partially straightened by the ring. The spikes may then be torn, as described before, for example adjacent a thickening 1254 in the spikes, at which time, the spikes will revert to their curved shape, as shown in FIG. 12J. In the curved configuration, the tips of the spikes are less likely to fall off ring 1256 and/or may apply a greater sealing pressure. Alternatively or additionally, the use of a curved tip reduces the presence of sharp points outside the blood vessel. In an exemplary embodiment of the invention, the curled spikes define an arc of over 180°, over 200°, over 270°, over 360° or any greater, smaller or intermediate arc angle.

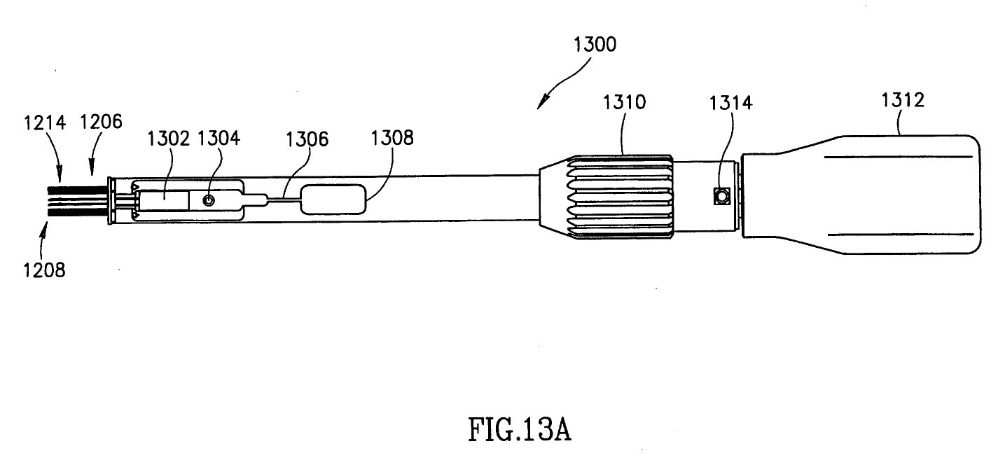

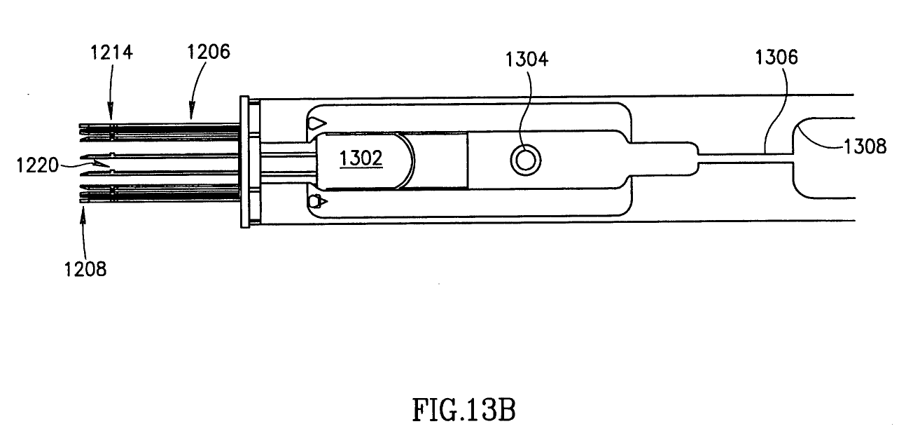

FIGS. 13A and 13B show a connector delivery system 1300, in accordance with an exemplary embodiment of the invention. Unlike delivery system 800, which is operated by lever 1000, system 1300 is operated by rotation of a knob 1312 relative to a handle 1310, so that an inner tube 1302, coupled to a plurality of puller spikes 1206, is retracted. In an exemplary embodiment of the invention, inner tube 1302 is threaded to match a thread on knob 1312.

As described in FIG. 12, puller spikes 1206 may include transaxial extensions 1214 and weakening 1220. Tips 1208 of the spikes may be bent, unlike shown in FIG. 12, for example as described in PCT/IL01/00074, by means of a jig that holds the spikes bent while they are heated and/or bends the spikes past a super-elastic memory point. Optionally, spikes 1206 are arranged in the shape of a cone, as are spikes 810 (FIG. 8), however, this is not required.

In an exemplary embodiment of the invention, system 1300 is used in peripheral vessels, where clamping of the vessel, to prevent blood leakage, is less problematic than in the heart.

In an exemplary embodiment of the invention, delivery system 1300 is a split system including a slit or weakening 1306 that is split by a protrusion 1304, when inner tube 1302 is retracted and protrusion 1304 enters or cuts slit 1306. An aperture 1308 may be used for providing the graft.

Optionally, one or more pins 1314 are provided. In an exemplary embodiment of the invention, pin 1314 is spring loaded and falls back to lock the rotation of knob 1312 when inner tube 1302 is retracted by an amount corresponding to a stage in the anastomosis procedure. In one example, pin 1314 overlies a plurality of holes in inner tube 1302. When tube 1302 is retracted to the point where extensions 1214 are locked against ring 1218, a first stage is completed. When weakenings 1220 are tom, a second stage is completed. When slit 1306 is widened, a third stage is completed. Alternatively, other feedback mechanism may be provided, for example, clicks in the rotating mechanism.

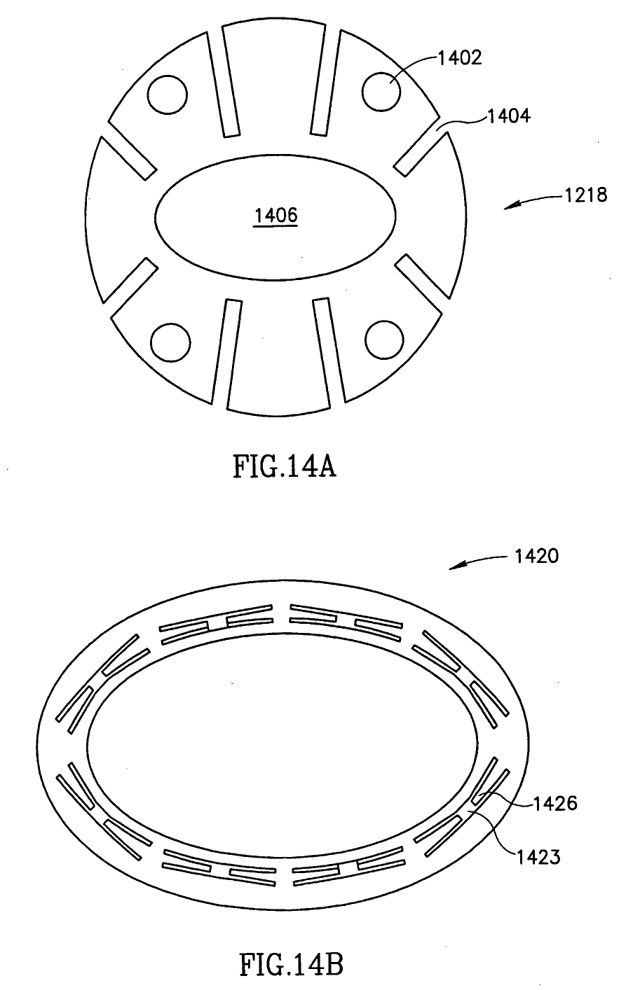

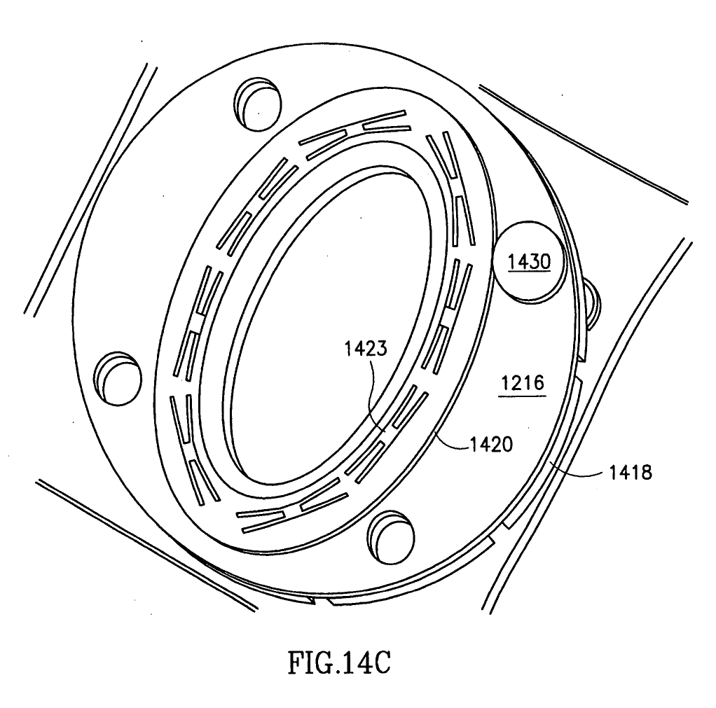

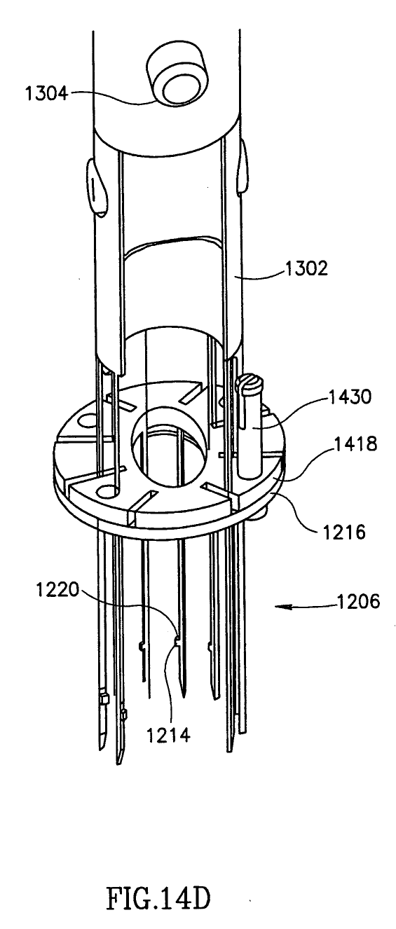

FIGS. 14A-14D illustrate a pair of interacting rings and their use in the system of FIG. 13. FIG. 14A shows an exemplary ring 1418, comprising a plurality of slots 1404 for inserting puller spikes 1206. Optionally, the spikes are twisted or bent radially, since the width of slots 1404 is smaller than that of extensions 1220. A central aperture 1406 is provided for passing the graft. A plurality of openings 1402 are optionally provided for inserting a spacer to separate ring 1218 from ring 1202 and/or for attaching ring 1218 to delivery system 1300, so it can be retracted with inner tube 1302 after puller spikes 1206 are tom.

FIG. 14B shows an exemplary connector ring 1420, as an alternative to ring 1202 of FIG. 12A. In an exemplary embodiment of the invention, a plurality of apertures 1423 are defined in the ring and correspond to slots 1404 in ring 1418. Two or more metal flaps 1426 optionally flank each hole, to allow extensions 1220 to be brought through apertures 1423.

FIG. 14C shows ring 142 and ring 1418, coupled together and spaced apart by spacer ring 1216, for example, a ring with a large central aperture. An exemplary bolt 1430 is shown coupling ring 1418 and spacer 1216. Optionally, ring 1420 is held in place between the heads of bolts 1430. Optionally, ring 1420 includes a cut-out, to assist aligning ring 1418 with ring 1420.

FIG. 14D shows spikes 1206 mounted through rings 1418 and 1216.

One potential problem with devices 800 and 1300 is that when the spikes are inserted into the target vessel, they may entangle each other and then will fail to retract and expand correctly.

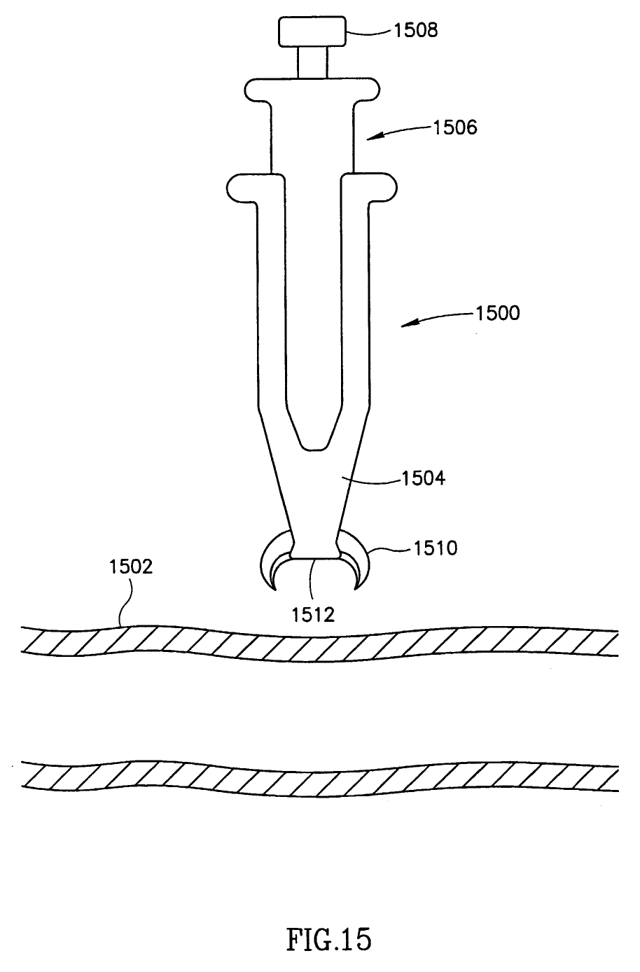

FIG. 15 illustrates an exemplary spike guiding device 1500, for inserting spikes into a target vessel 1502. Device 1500 comprises generally of a cone-shaped body 1504, which is optionally split lengthwise. Optionally, a small inverse pointed cone 1512 is defined at its tip, to assist in fixing body 1504 in target vessel 1502.

For the purpose of cutting a hole in target vessel 1502, a cutting mechanism 1506 is inserted into the guide. In an exemplary embodiment of the invention, the cutting mechanism includes a plunger 1508, that when advanced, causes two cutting heads 1510 (e.g., hooks) to turn towards each other and engage and cut vascular tissue between them. In an exemplary embodiment of the invention, body 1504 has a split head and the cutting heads pass through the split.

While a short device is shown, a similar, longer, device may be used for thoracic or trans-vascular use device 1500 maybe flexible or rigid.

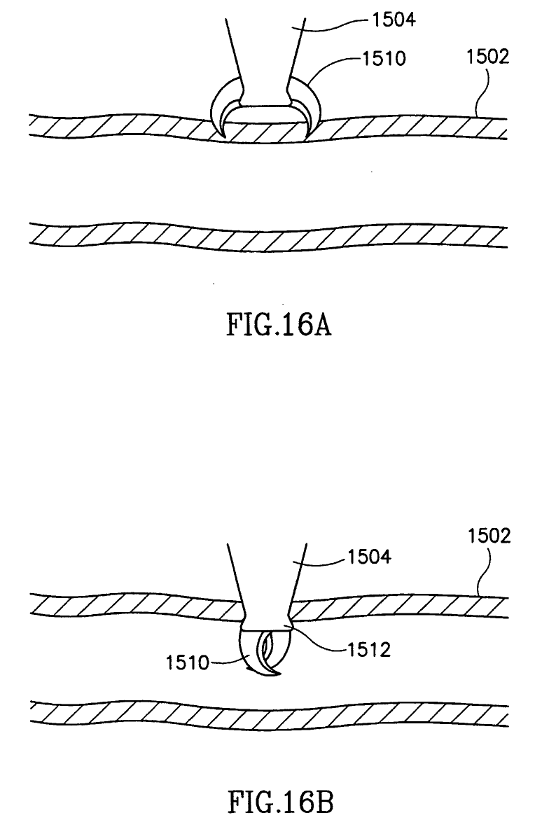

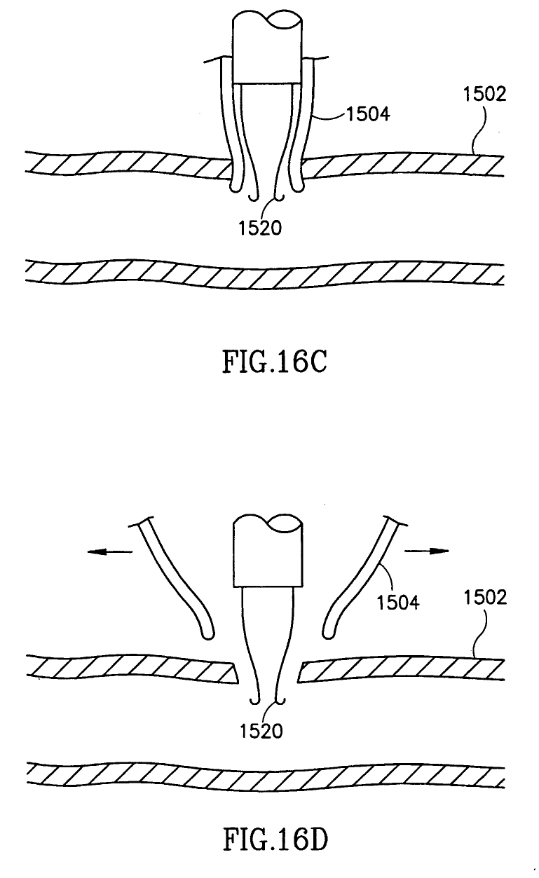

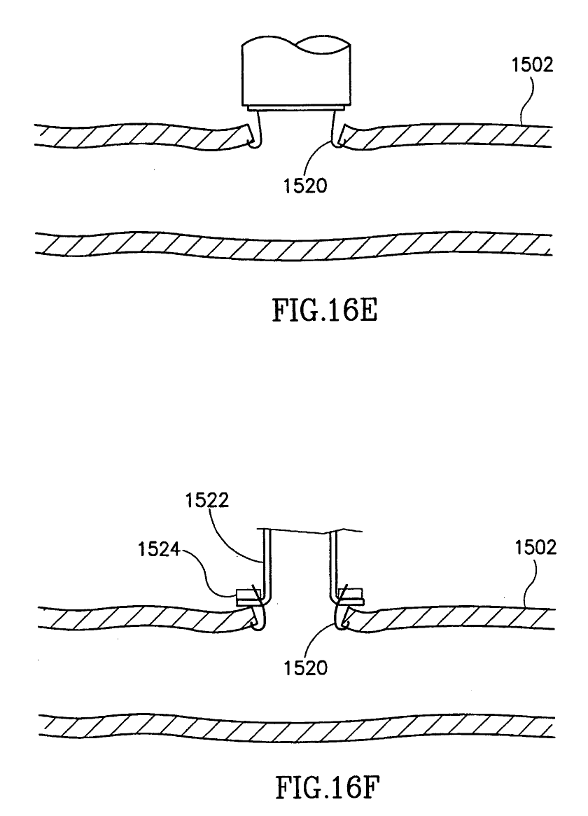

FIGS. 16A-16F illustrate a process of performing an anastomosis using guide device 1500, in accordance with an exemplary embodiment of the invention. In FIG. 16A, body 1504 contacts target vessel 1502 and cutting heads 1510 start to close so they engage the target vessel.

In an exemplary embodiment of the invention, the target vessel is a coronary vessel, in which cutting, rather than punching is desired. Alternatively, punching, or a different method of cutting than shown, may be used.

In FIG. 16B, body 1504 is advanced, so that inverse cone 1512 is inside the vessel. Optionally, cutting heads 1510 pull body 1504 towards target vessel 1502 and/or pull out the wall of target vessel 1502. In one example, heads 1510 are pointed outward, rather than inward as shown.

In FIG. 16C, cutting mechanism 1506 is removed and is replaced by a delivery system (e.g. 800) having a plurality of forward spikes 1520 mounted on a graft 1522 (FIG. 16F). Optionally, when the delivery system is advanced towards target vessel 1502, guide 1504 widens to accommodate it, thus widening the opening formed in target vessel 1502 so that spikes 1520 do not tangle. Optionally, guide 1504 is filled with a water soluble gel, to reduce or prevent blood leakage.

In FIG. 16D, guide 1504 is removed, for example being pulled back or torn into two. Optionally, guide 1504 is formed of two (or more) parts to begin with so it can simply be removed in parts.

In FIG. 16E, spikes 1520 are retracted, forming an anastomosis.

FIG. 16F shows a completed anastomosis, in which a graft 1522 is coupled to target vessel 1502, by a connector comprising spikes 1520 and a ring 1524 (e.g., as shown in FIG. 12).

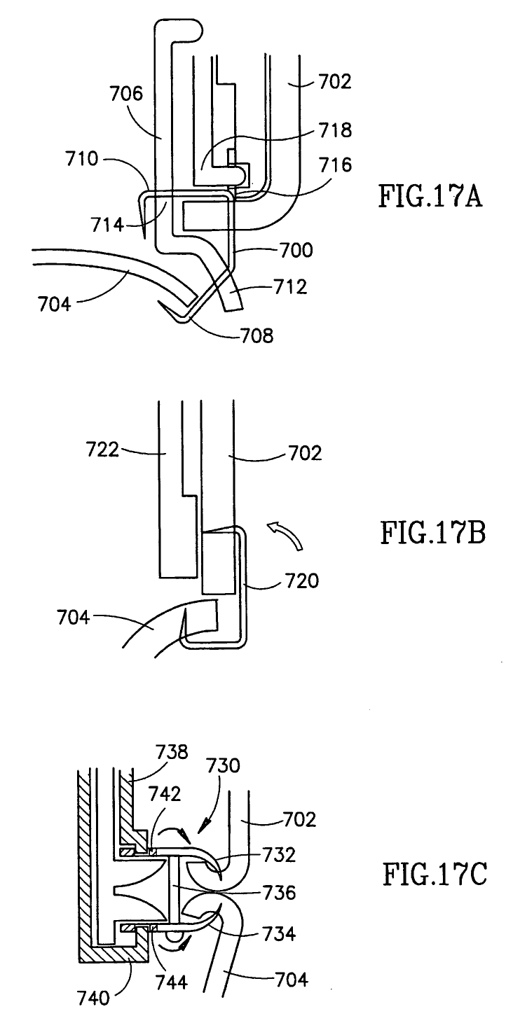

FIGS. 17A-17C illustrate the deployment of other clip-devices for the attachment of two blood vessels, in accordance with exemplary embodiments of the invention.

In FIG. 17A, a clip 700 is elastic, super-elastic of shape memory, so that it desires to reach a folded shape (i.e., is self-closing). A restraint 706, including, for example a slotted portion 712 that engages an arm 708 of clip 700, prevents the arm from closing. Another arm 710 of clip 700 may be held in another slot (or bump) 714. Alternatively, clip 700 is part of a single connector including a plurality of clips attached to a ring 716, here shown being held by a holder 718.

In operation, the lip of a graft 702 is transfixed by arm 708 of clip 700. Arm 708 is inserted into an incision in a vessel 704 (only one side shown). When restraint 706 releases clip 700, the clip closes, sealing together graft 702 and vessel 704. In a plastically deformed embodiment, a retraction of restraint 706 may fold arm 708 against arm 710.

FIG. 17B shows an alternative embodiment of the invention, in which a self-closing clip 720 transfixes a graft 702 and a vessel 704. The clip maybe inserted, for example, manually, into an incision in vessel 704 and then embedded in the vessel wall by pulling it back. When a restraining outer tube 722 is retracted, clip 720 is free to fold. Like clip 700, also clip 720 may be part of a multi-clip connector, in some embodiments of the invention.

FIG. 17C shows an alternative embodiment of the invention, in which a clip 730 has the lips of graft 702 and vessel 704 inserted into it, for example manually or using a puller (not shown). Clip 730 comprises two arms 732 and 734 connected by a base 736. In an exemplary embodiment of the invention, clip 730 is pre-stressed so that arms 732 and 734 desire to fold inwards. Arms 732 and 734 extend past base 736 as extensions 742 and 744, respectively. A restraint comprising two opposing restraint elements 738 and 740 engage and maintain in position, clip 730, via extensions 742 and 744. Clip 730 is a self-closing clip, in which arms 732 and 734 close towards each other. When restraints 738 and 740 are brought apart, arms 732 and 734 advance towards each other and engage and seal together the lips of graft 702 and vessel 704. In an exemplary embodiment of the invention, restraints 738 and 740 are brought together in order to enlarge the distance between arms 732 and 734 and make it easier to insert the lips of vessel 704 and graft 702 into the clip. Possibly, clip 730 is laid against the vessel and the graft and as the restraints are let apart, the tips of arms 732 and 734 engage and advance the lips into the clip.

The above devices may be varied in various ways, for example for adaptation for specific types of blood conduits. In some embodiments of the invention, a device is packaged and/or sold with an instruction leaflet, describing the device dimensions and/or situations for which the device should be applied.

One or more of the following parameters of a device may be varied, for example:

(a) Number of barbs in an arm of a clip. Although only one barb is shown, two, three or more barbs may be provided.

(b) Location of barbs along the arm. Although the barbs are shown at the tip of the arm, they may be positioned further in. In devices with multiple barbs, the barbs may be positioned side by side or one in front of the other, for example.

(c) Shape of arms. Various shapes may be provided, for example, rectangular, triangular, arcuate, circular and piecewise linear or curved. The arms may be planar or may extend outside of a plane, for example being curved.

(d) Length of barbs. The barbs may be long enough to transfix the vessel walls. Alternatively, they may be made shorter, for example penetrating only some of the layers of the blood vessel. It is noted that different barbs on a same device may have different lengths or other properties, for example, depending on the properties of the target vessels. Exemplary lengths include, 0.1 mm, 0.5 mm, 1 mm, 2 mm and larger, smaller and intermediate sizes. Other parameters of the barb design may vary as well, for example the degree of sharpness (sharp vs. blunt).

(e) Length of arms. The length of the arms may also depend on the properties of the target vessels and the geometry of the connection. Exemplary lengths include, 1 mm, 3 mm, 5 mm, 7 mm and larger, smaller and intermediate sizes.

(f) Existence and dimensions of base. Although not all devices include a base, the length of the base may be, for example, 1 mm, 3 mm, 5 mm, 7 mm and larger, smaller and intermediate sizes. The width of the base (between two arms) may be, for example, 0.5 mm, 1 mm, 3 mm, 5 mm and larger, smaller and intermediate sizes.

(g) Existence and geometry of lumen. In devices with a central lumen, the shape of the lumen and the punched hole may be vary, for example being circular, elliptical or polygonal.

(h) Solidity of device. Although the device may have a continuous surface, in some embodiments of the invention, for example as shown in FIGS. 3A and 1E, one or more holes may be formed in the surface of the device. This may reduce the total amount of foreign material in the body. It is noted, however, that the total amount of material in the blood flow may be very low or even zero, in some embodiments of the invention.

(i) Smoothness. For devices that attach two vessel parts by pressure, the means applying the pressure (e.g., fingers 754) may be smooth. Alternatively, they may be bumpy, rough or include small spikes or barbs. Optionally, a large pattern is defined. For example, the fingers 754 may match indentations in ring 752.

It will be appreciated that the above described methods and devices of vascular manipulation may be varied in many ways, including, changing the order of steps, which steps are performed inside the body and which outside, the order of making the anastomosis connections, the order of steps inside each anastomosis, the exact materials used for the anastomotic connectors, which vessel is a “side” side and which vessel (or graft) is an “end” side of an end-to-side anastomosis and/or whether two lips that are connected are from a same vessel or from different vessels. Further, in the mechanical embodiments, the location of various elements may be switched, without exceeding the sprit of the disclosure, for example, switching the moving elements for non-moving elements where relative motion is required. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of the above features, from different described embodiments are also considered to be within the scope of some exemplary embodiments of the invention. In addition, some of the features of the invention described herein may be adapted for use with prior art devices, in accordance with other exemplary embodiments of the invention. The particular geometric forms used to illustrate the invention should not be considered limiting the invention in its broadest aspect to only those forms, for example, where a circular lumen is shown, in other embodiments an oval lumen may be used.

Also within the scope of the invention are surgical kits which include sets of medical devices suitable for making a single or a small number of anastomosis connections. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A vascular attachment device for sealing an opening between two blood conduit lips, comprising:
a ring element;
a plurality of fingers mounted on said ring element and adapted to seal at least a portion of an opening between two blood conduit lips by compressing said at least two lips between a finger and at at least one of said finger and said ring; and
at least one puller spike adapted for pulling, inside the body, at least one of said lips to a space defined between said finger and said ring.

2. A device according to claim 1, wherein said fingers are restrained back from a resting position in which they engage said lip in said space.

3. A device according to claim 1, wherein said at least one puller is integral with an elongate retractable tube.

4. A device according to claim 1, wherein said at least one puller comprises a plurality of pullers arranged in the form of a cone, an apex of said cone being adapted for inserting into an opening in a blood vessel.

5. A device according to claim 4, wherein said at least one puller is outside of said ring such that when said pullers are retracted, the cone opens up.

6. A device according to claim 4, wherein each puller comprises a bent tip, adapted to engage said lip.

7. A device according to claim 1, wherein each puller comprises a designated tear area, for tearing said puller after it is retracted towards said ring, so that only a tip portion of said puller remains in the body.

8. A device according to claim 7, wherein said tip portion is pre-curled such that said tearing allows said portion to revert to a curled closed state.

9. A device according to claim 7, wherein each puller comprises a trans-axial protrusion for stopping retraction of said tip portion.

10. A device according to claim 1, wherein each puller is smooth, to allow retraction of said puller through said lips and out of said body.

11. A device according to claim 1, wherein each puller has a sharp tip adapted for insertion through a graft wall.

12. A device according to claim 1, wherein said ring has the shape of an ellipse.

13. A device according to claim 1, wherein said fingers do not penetrate any of said lips.

14. A vascular attachment device for sealing an opening between two blood conduit lips, comprising a plurality of bendable clips, said clips being adapted for gripping two lips between them and for sealing said opening by forcing said lips towards each other, wherein said clip elements are blunt and do not penetrate said blood conduit walls.

15. A device according to claim 14, wherein said clips are arranged on a ring.

16. A vascular attachment device for sealing an opening between two blood conduit lips, comprising:
a ring element defining a plurality of apertures;
a plurality of puller spikes having tips and defining designated tear areas near said tips, said tips being adapted to fit through said apertures and integral with a retractable elongate tube, such that when said device is deployed only said tips of said spikes remain in said body.

17. A device according to claim 16, wherein said tube is an axially split tube.

18. A device according to claim 16, wherein said tube comprises a protrusion adapted for axially splitting a matching delivery system, when said tube is sufficiently retracted.

19. A device according to claim 16, wherein said spikes are pre-curled, such that when said spiked are torn at said designated tear areas, said tips revert to a pre-curled state having a greater curl arc angle than prior to said tearing.

20. A device according to claim 16, wherein said apertures define leaf elements for preventing reverse motion of said spikes.

21. A device according to claim 16, wherein said spikes define a protrusion on said spikes adjacent said designated tear areas.

22. A connector delivery system, comprising:
a retractor;
a tube integral with a plurality of puller spikes of said connector, said tube coupled to said retractor for retraction thereby, said spikes defining at least one thickened areas on at least one spike; and
a base ring for preventing said at least one thickened areas from retracting, thereby causing said spikes to tear when said retractor retracts said tube a sufficient amount.

23. A system according to claim 22, wherein said tube comprises a protrusion and wherein said delivery system is adapted to be split by said protrusion when said tube is sufficiently retracted.

24. A system according to claim 22, comprising a stationary tube for maintaining said base ring in place relative to said integral tube.

25. A connector delivery system for delivering a ring connector having a plurality of fingers, said fingers defining an open configuration and a closed configuration mounted thereon, comprising:
a retractor
a tube integral with a plurality of puller spikes and coupled to said retractor for retraction thereby; and
an outer tube adapted to close a plurality of said fingers, when said puller spikes are retracted into said ring connector.

26. A system according to claim 25, wherein said outer tube defines a plurality of slots, for guiding a straightening of said puller spikes, when said puller spikes are retracted past said connector.

27. A system according to claim 25, wherein said fingers close plastically.

28. A system according to claim 27, wherein said outer tube has an inner lip with an inner diameter smaller than an outer diameter of said connector, such that when said outer tube is moved relative to said connector, said fingers are pushed inwards by the inner lip towards said ring.

29. A system according to claim 25, wherein said fingers close to said closed configuration by said fingers being released.

30. A system according to claim 29, wherein said outer tube defines an inner lip, against which said fingers are held away from said ring, such that when said outer tube is retracted, said fingers are released from said lip and close.

31. A system according to claim 29, wherein said outer tube defines a plurality of slots, said fingers being held in said slots, such that when said outer tube is retracted, said fingers are released from said slots and close.

32. A system according to claim 31, wherein said slots have a width said slot width being narrower than a width of said fingers.

33. A system according to claim 31, wherein said slot width is less than 10% narrower than said finger width.

34. A system according to claim 25, comprising a stationary tube for maintaining said connector in place relative to said integral tube.

35. A method of pulling back fingers of a ring connector, in preparation for performing an anastomosis connection, comprising:
providing a connector delivery system including a slotted outer tube, said connector being mounted inside said tube, such that said fingers match up with said slots;
inserting a tool into a slot to be guided by said slot and to contact said finger; and
pulling back said fingers using said tool, to be held by said slotted outer tube.

36. A method according to claim 35, wherein said fingers are pulled back to lie in said slots.

37. A method according to claim 35, wherein said fingers are pulled back using a tool inserted through said slots, such that said fingers lie within an inner lip of said outer tube.

38. Apparatus for anastomosis, comprising:
a delivery system includes conical shaped arrangement of puller spikes;
a cone shaped body defining an opening at either end, a wide opening, at abase thereof, for receiving said conical arrangement and a narrow opening, at an apex thereof, for insertion into a blood vessel.

39. Apparatus according to claim 38, wherein said cone shaped body is so shaped that when said delivery system is advanced, said narrow opening widens.

40. Apparatus according to claim 38, wherein said cone shaped body is pre-split axially.

41. Apparatus according to claim 38, comprising a cutting mechanism adapted to fit in said cone and comprising at least one cutting blade that fits through said narrow opening said cone.

42. Apparatus according to claim 41, comprising a smaller, base-first cone having an apex meeting said apex of said cone, for inserting in a hole cut by said at least one blade, such that said smaller cone and said cone define a saddle.

43. Apparatus according to any of claims 38-42, wherein said cone is at least partially pre-split from an apex thereof.

44. Apparatus according to claim 43, wherein said cone is pre-split on opposite sides.
